(19) United States
(12) Patent Application Publication
(10) Pub. No.: US 2009/0246779 A1
(43) Pub. Date: Oct. 1, 2009

(54) INCREASING GENOMIC INSTABILITY DURING PREMALIGNANT NEOPLASTIC PROGRESSION REVEALED THROUGH HIGH RESOLUTION ARRAY-CGH

(75) Inventors: Peter S. RABINOVITCH, Shoreline, WA (US); Lisa A. LAI, Seattle, WA (US)

Correspondence Address: NIXON PEABODY LLP - PATENT GROUP, 1100 CLINTON SQUARE, ROCHESTER, NY, 14604, US

(73) Assignee: UNIVERSITY OF WASHINGTON, Seattle, WA (US)

(21) Appl. No.: 12/372,392

(22) Filed: Feb. 17, 2009

Related U.S. Application Data
(60) Provisional application No. 61/029,123, filed on Feb. 15, 2008.

(51) Int. Cl. C12Q 1/68; G01N 33/48

(52) U.S. Cl. 435/6; 436/94

(57) ABSTRACT

The present invention relates to a method of diagnosing a premalignant condition based on the presence of chromosomal alterations at one or more fragile chromosome sites. Methods of determining chromosomal alterations are disclosed. The present invention also relates to methods of establishing a prognosis and a therapeutic regimen for the subject having the premalignant condition are also disclosed.

This application claims the benefit of U.S. Provisional Patent Application Ser. No. 61/029,123, filed Feb. 15, 2008, which is hereby incorporated by reference in its entirety.

This invention was made with government support under grant number P01 CA 091955 awarded by National Institutes of Health. The government has certain rights in this invention.

### FIELD OF THE INVENTION

The present invention relates to molecular diagnostics for the diagnosis and prognosis of premalignant conditions based on chromosomal fragile site stability.

### BACKGROUND OF THE INVENTION

Fragile sites are loci that exhibit an increased propensity for sister chromatid exchange, translocation, and breaks under conditions of genotoxic stress (Yunis et al., “Constitutive Fragile Sites and Cancer,” Science 226:1199-204 (1984), and Sutherland et al., “The Molecular Basis of Fragile Sites in Human Chromosomes,” Curr Opin Genet Dev 5:323-7 (1995)). The susceptibility of these loci to damage is believed to be a consequence of their primary and secondary structure, which affects chromatin organization and ultimately stalls DNA replication (Wang Y. H., “Chromatin Structure of Human Chromosomal Fragile Sites,” Cancer Lett 232:70-8 (2006) and Schwartz et al., “The Molecular Basis of Common and Rare Fragile Sites,” Cancer Lett 232:13-26 (2006)). The resulting DNA gaps, breaks, and other chromosomal aberrations at fragile sites impact genomic stability, and often manifest as deletions and translocations. Currently, there are over one hundred documented fragile sites within the human genome, categorized as “common” (present in all individuals) or “rare” (present in less than 5% of the population) (Wang Y. H., “Chromatin Structure of Human Chromosomal Fragile Sites,” Cancer Lett 232:70-8 (2006) and Schwartz et al., “The Molecular Basis of Common and Rare Fragile Sites,” Cancer Lett 232:13-26 (2006)); most are defined cytogenetically and their molecular characterization is not known.

While instability at specific fragile sites has been linked to different cancers (O'Keefe et al., “Common Chromosomal Fragile Sites and Cancer: Focus on FRA16D Common Chromosomal Fragile Site Oxido-Reductase (FOR/WWOX) Protects Against the Effects of Ionizing Radiation in Drosophila,” Cancer Lett 232:37-47 (2006)) including breast (Ahmadian et al., “Analysis of the FHIT Gene and FRA3B Region in Sporadic Breast Cancer, Preneoplastic Lesions, and Familial Breast Cancer Probands,” Cancer Res 57:3664-8 (1997)), prostate, and lung (Fong et al., “FHIT and FRA3B 3p14.2 Allele Loss Are Common in Lung Cancer and Preneoplastic Bronchial Lesions and Are Associated With Cancer-Related FHIT cDNA Splicing Aberrations,” Cancer Res 57:2256-67 (1997) and Nymark et al., “Identification of Specific Gene Copy Number Changes in Asbestos-Related Lung Cancer,” Cancer Res 66:5737-43 (2006)), there is still uncertainty as to whether these fragile site alterations causally contribute to cancer development or are merely “silent markers” of genomic stress. Putative tumor suppressors have been suggested to be located within common fragile sites; the fragile histidine triad gene (FHIT) at FRA3B and the WW-domain containing oxidoreductase (WWOX) at FRA16D have the best evidence for a role in cancer progression (Kuroki et al., “Common Fragile Genes and Digestive Tract Cancers,” Surg Today 36:1-5 (2006)), while most other genes known to be at fragile sites, such as parkin at FRA6E have less clear evidence for roles as tumor suppressors (Smith et al., in Cancer Lett 48-57 (2006)). Alternatively, breakage at fragile sites could contribute to repeated cycles of bridge-breakage-fusion, potentially promoting the amplification of oncogenes (Hellman et al., “A Role For Common Fragile Site Induction in Amplification of Human Oncogenes,” Cancer Cell 1:89-97 (2002)) such as Met within the FRA7G region (Miller et al., “Genomic Amplification of MET With Boundaries Within Fragile Site FRA7G and Upregulation of MET Pathways in Esophageal Adenocarcinoma,” Oncogene 25:409-18 (2006)) or the prolactin-inducible protein (PIP) gene (Ciullo et al., “Initiation of the Breakage-Fusion-Bridge Mechanism Through Common Fragile Site Activation in Human Breast Cancer Cells: the Model of PIP Gene Duplication From a Break at FRA7I,” Hum Mol Genet 11:2887-94 (2002)).

Barrett's esophagus (BE) is an excellent model system in which to develop and study biomarkers of cancer risk and to study mechanisms of neoplastic progression; this pre-malignant condition is the only known precursor of esophageal adenocarcinoma (EA) and develops in the context of chronic gastro-esophageal reflux disease, with repeated cycles of injury and repair from bile, acid, and inflammation (Sihvo et al., “Oxidative Stress Has a Role in Malignant Transformation in Barrett's Oesophagus,” Int J Cancer 102:551-5 (2002) and Jenkins et al., “Genetic Pathways Involved in the Progression of Barrett's Metaplasia to Adenocarcinooma,” Br J Surg 89:824-37 (2002)). To date, molecular markers including abnormal DNA content and loss of heterozygosity (LOH), mutation, or methylation at p16 and p53, have been shown to be predictive of the risk of disease progression (Wong et al., “p16(INK4a) Lesions Are Common, Early Abnormalities That Undergo Clonal Expansion in Barrett's Metaplastic Epithelium Evolution of Neoplastic Cell Lineages in Barrett Oesophaguis,” Cancer Res 61:8284-9 (2001); Reid et al., “Predictors of Progression in Barrett's Esophagus II: Baseline lip (p53) Loss of Heterozygosity Identifies a Patient Subset At Increased Risk For Neoplastic Progression,” Am J Gastroenterol 96:2839-48 (2001); Galipeau et al., “Clonal Expansion and Loss of Heterozygosity At Chromosomes 9p and 17p in Premalignant Esophageal (Barrett's) Tissue,” J Natl Cancer Inst 91:2087-95 (1999); and GalTipeau et al., “NSAIDs Modulate CDKN2A, TP53, and DNA Content Risk For Progression to Esophageal Adenocarcinoma,” PLoS Med 4:e67 (2007)). However, the frequency, degree, and specificity of chromosomal instability and how it may contribute to cancer development is unknown.

The present invention is directed to overcoming these and other deficiencies in the art.

### SUMMARY OF THE INVENTION

A first aspect of the present invention relates to method of diagnosing a premalignant condition in a human subject. This method includes providing a genomic DNA sample from a human subject and determining the presence of chromosomal alterations at one or more fragile chromosome sites in the genomic DNA sample. The one or more fragile chromosome sites to be analyzed include, but are not limited to, FRA13B, FRA16D, FRA1K, FRA11D, FRA12B, FRA20A, FRA10C, FRA10D, FRA7I, FRA9A/9C, FRA4D, FRA5E, FRAXC, and FRA18C. This method also includes diagnosing the premalignant condition, based on the presence of chromosomal alteration at one or more of the above fragile chromosome sites.

A second aspect of the present invention includes a diagnostic kit which contains a detection assay for determining, in a DNA sample from a human subject, chromosomal alterations at one or more sites within one or more fragile chromosome sites. The fragile chromosome sites include FRA13B, FRA16D, FRA1K, FRA11D, FRA12B, FRA20A, FRA10C, FRA10D, FRA7I, FRA9A/9C, FRA4D, FRA5E, FRAXC, and FRA18C.

A third aspect of the present invention relates to a method of diagnosing a premalignant condition in a human subject, This method includes providing a genomic DNA sample from a human subject and determining the presence of a genomic deletion at the FRA3B fragile site. The genomic deletion at the FRA3B fragile site comprises a 250 kb region of the FHIT gene and includes at least a portion of intron 4 and at least a portion of intron 5 of the FHIT gene. The method further includes diagnosing the premalignant condition based on the presence of the genomic deletion at the FRA3B fragile site.

A fourth aspect of the present invention includes a diagnostic kit which contains a detection assay for determining, in a DNA sample from a human subject, a genomic deletion in the FRA3B fragile site. The genomic deletion comprises a 250 kb region of the FHIT gene including at least a portion of intron 4 and at least a portion of intron 5 of the FHIT gene.

Applicants hypothesize that DNA damage resulting from inflammation, reactive oxygen species, and cycles of cellular injury and repair in premalignant conditions could specifically promote replicative stress and breakage at chromosome fragile sites. In a whole genome analysis, of a diverse sampling of Barrett Esophagus patient specimens, genomic instability (copy loss and/or LOH) was found at many fragile sites, some of which demonstrate increasing alterations with disease progression. The high frequency of copy loss and LOH within defined regions of multiple chromosomal fragile sites can be utilized to develop sensitive molecular diagnostic tests for the detection of BE and other premalignant conditions having a similar etiology.

### BRIEF DESCRIPTION OF THE DRAWINGS

FIGS. 1A-B are a comparison of chromosome copy number plots generated from a BAC array (FIG. 1A) or the Affymetrix 50K SNP array (FIG. 1B) for a single chromosome, chromosome 5, from the CDKN2A^{LOH}/TP53^{LOH}/aneuploid sample for Patient 1. The copy number estimates are for paired BE and lymphocyte constitutional samples. Gains are represented by points above log₂ ratio=0 and losses are shown by points below log₂ ratio=0. The insets show magnifications of the regions indicated with arrows. The SNP array discriminates many smaller copy gain and loss events that are below the resolution of the BAC array.

FIGS. 2A-C show the increasing number and size of regions of copy number gain, copy number loss, and LOH with neoplastic progression for three patients (1, 2, and d). Shown are histograms of the number of regions (ordinate) with copy number gain (left column), loss (middle column), or LOH (right column) in BE samples as a function of size of alteration (abscissa, bp). Data for patient 1, 2, and 3 is represented by FIGS. 2A, 2B, and 2C respectively. Samples represent the three molecular stages of BE, i.e., CDKN2A^{LOH} diploid; diploid CDKN2A^{LOH}/TP53^{LOH}; and CDKN2A^{LOH}/TP53^{LOH} with aneuploidy.

FIG. 3 is a consensus plot of copy number alterations for early BE samples. Shown is a genome-wide consensus plot generated by the Cluster Along Chromosomes (CLAC) program for CDK2A^{LOH} only BE samples analyzed on Affymetrix 50K arrays. Chromosome numbers are indicated on the left side and the horizontal lines represent the baselines (log₂ ratio=0) for each chromosome. The centromere positions are indicated by the single vertical line. The bars above baseline represent copy gains and the lines below baseline represent copy loss. The height of the line reflects the number of samples which displayed the alteration (see bottom inset). The three top insets magnify three regions of copy number loss at FHIT/FRA3B, CDKN2A, and FRA13B, within chromosome bands 3p14, 9p21, and 13q22, respectively, which were detected in the majority of cases tested.

FIG. 4 depicts the evolution of SNPs analyzed as copy number gains, copy number losses, or LOH. The Venn diagrams indicate the distribution of SNPs called as gains (top row), losses (middle row), or LOH (bottom row) for Patients 1 (left column), 2 (middle column), and 3 (right column). Samples were (clockwise from top left) (A) diploid CDKN2A^{LOH}, (B) diploid CDKN2A^{LOH}/TP53^{LOH}, and (C) aneuploid CDKN2A^{LOH}/TP53^{LOH}, with overlapping numbers of events as indicated. Gains and losses were determined using the CLAC program. Genotype calls were outputted from Affymetrix GeneChip Genotyping Analysis Software (GTYPE) version 4.1 and analyzed as described infra.

FIG. 5 shows the copy loss (top), copy gain (middle), and LOH (bottom) at fragile sites in early BE samples. Shown is frequency of copy loss, copy gain, or LOH within the respective cytoband regions for each fragile site. Copy number changes were determined using the CGH-Miner software and LOH using the relative allele frequency between BE sample and paired constitutional sample.

FIG. 6 is an Affymetrix 100K array analysis of the FHIT gene in six early stage (p16^{LOH} only) BE patients, showing the consensus region of loss in FHIT exon 5 at the 5′ end of FRA3B. Normal (two) gene copies corresponds to log 2 ratio=0; losses are indicated below zero and amplifications above zero. One patient (top line) has trisomy in the flanking regions, reduced by loss to 2 copies in FRA3B.

FIGS. 7A-B show the PCR strategy for detecting the FRA3B deletion. In FIG. 7A, primers are shown flanking the region of deletion. In the absence of a deletion, the primers span a distance of ˜250 kb, too large to be amplified by PCR. However, in the event of deletion, the primers are in close proximity (<30 kb) and a product is obtained. FIG. 7B shows primer pairs spaced apart at different distances along the regions upstream and downstream of the FRA3B deletion to detect FRA3B deletions of variable size.

FIG. 8 shows PCR products generated using long extension PCR across FRA3B. The products were generated with one forward primer (2F) (SEQ ID NO: 111) and staggered reverse primers (3R-7R) (SEQ ID NOs: 112-116) which flank the fragile site. The control male DNA was purchased from Promega and QhTRT is a previously characterized BE cell line (Palanca-Wessels, et al., “Extended Lifespan of Barrett's Esophagus Epithelium Transduced with the Human Telomerase Catalytic Subunit: A Useful In Vitro Model,” Carcinogenesis 24(7):1183-90 (2003), which is hereby incorporated by reference in its entirety).

### DETAILED DESCRIPTION OF THE INVENTION

A first aspect of the present invention relates to method of diagnosing a premalignant condition in a human subject. This method includes providing a genomic DNA sample from a human subject and determining the presence of chromosomal alterations at one or more fragile chromosome sites in the genomic DNA sample. The one or more fragile chromosome sites to be analyzed include, but are not limited to, FRA13B, FRA16D, FRA1K, FRA11D, FRA12B, FRA20A, FRA10C, FRA10D, FRA7I, FRA9A/9C, FRA4D, FRA5E, FRAXC, and FRA18C. This method also includes diagnosing the premalignant condition, based on the presence of chromosomal alteration at one or more of the above fragile chromosome sites.

In accordance with this aspect of the present invention, the method of diagnosing a premalignant condition in a human subject can further involve determining the presence of a genomic deletion at the FRA3B fragile chromosome site. Although the detection of any genomic deletion at the FRA3B fragile chromosome site will have diagnostic utility, in a preferred embodiment, the genomic deletion at the FRA3B fragile site comprises a 250 kb region of the FHIT gene. This 250 kb region includes at least a portion of intron 4 of the FHIT gene, and at least a portion of intron 5 of the FHIT gene.

A second aspect of the present invention relates to a method of diagnosing a premalignant condition in a human subject. This method includes providing a genomic DNA sample from a human subject and determining the presence of a genomic deletion at the FRA3B fragile site. The genomic deletion at the FRA3B fragile site comprises a 250 kb region of the FHIT gene and includes at least a portion of intron 4 and at least a portion of intron 5 of the FHIT gene as described supra. The method further includes diagnosing the premalignant condition based on the presence of the genomic deletion at the FRA3B fragile site.

As used herein, a “premalignant condition” refers to a tissue that is not yet malignant or cancerous, but is poised to become malignant or cancerous. A premalignant condition be can associated with or arise in any tissue of the body, including, but not limited to, prostate, gastrointestinal, breast, lung, skin, cervical, esophageal, bone, pancreatic, oral, or bladder tissue. Examples of premalignant conditions include, but are in no way limited to, colon polyps, pernicious anemia, atrophic gastritis, Barrett's esophagus, actinic keratosis, ductal carcinoma in situ, cervical dysplasia, endometrial hyperplasia, squamaous metaplasia, leukoplakia, and erythroplakia. In a preferred embodiment of the present invention, the premalignant condition is Barrett's esophagus. The premalignant condition to be diagnosed by the methods of the present invention may be caused by or associated with conditions of chronic inflammation or reactive oxygen species.

The method of the present invention involves determining the presence of chromosomal alterations at one or more fragile sites in a genomic DNA sample. According to the present invention, a chromosomal alteration encompasses any variation in the genomic DNA nucleic acid sequence, including, but not limited to, differences in the lengths of repeated sequence elements, such as minisatellites and microsatellites, small insertions or deletions, and single nucleotide polymorphisms (SNPs). Chromosomal alterations also include genomic copy number losses, resulting from deletions or replication failure at a chromosomal locus, or genomic copy number gains, resulting from duplication or multiplication events. According to the present invention, a chromosomal alteration also includes loss of heterozygosity (LOH) at a particular locus arising from deletion, mutational events, chromosomal rearrangements, gene conversion, mitotic recombination, or allelic imbalance due to selective amplification of one allele over the other.

In accordance with the methods of the present invention, genomic DNA can be isolated directly from cells or tissue associated with the premalignant condition. Preferably, the cells or tissue are harvested in a noninvasive manner. In a preferred embodiment, the genomic DNA sample can be obtained from a peripheral blood sample.

In the case of BE, the genomic DNA sample can be obtained from esophageal biopsy tissue or from epithelial cells associated with the premalignant condition. The cell sample can be a mixed cell sample containing BE and non-BE cells. It is preferred that the cells are collected using minimally invasive procedures such as using a cytological brush or an ultra thin fiber optic endoscopic instrument. Alternatively, the genomic DNA sample can be isolated from a peripheral blood sample.

Genomic DNA may be isolated by standard procedures that are readily available to those of skill in the art. Representative methodology are provided, for example, in Sambrook et al., Molecular Cloning: A Laboratory Manual (Cold Spring Harbor Laboratories, Cold Spring Harbor, N.Y., 1989) and in Ausubel et al. (eds.), Short Protocols in Molecular Biology (4th Ed., John Wiley & Sons 1999) which are hereby incorporated by reference in their entirety.

The methods of the present invention include investigating chromosomal integrity at one or more fragile sites. Fragile sites are chromosomal loci which exhibit an increased propensity for sister chromatid exchange, translocation, and breaks under stress conditions which inhibit DNA replication (Yunis and Soreng, “Constitutive Fragile Sites and Cancer,” Science 226:1199-1204 (1984), and Sutherland and Richards, “The Molecular Basis of Fragile Sites in Human Chromnosomes,” Curr Opin Genet Dev. 5:323-327 (1995), which are hereby incorporated by reference in their entirety). This phenomenon is a consequence of primary and secondary structure, resulting in poor chromatic organization or stalled DNA replication (Wang Y. H., “Chromatin Structure of Human Chromosomal Fragile Sites,” Cancer Lett. 232:70-78 (2006), which is hereby incorporated by reference in its entirety). These DNA gaps and subsequent breaks result in recombination events, thereby affecting genomic stability often times through copy number loss (deletion) of genomic sequence. There are over one hundred documented fragile sites within the human genome separated into “common” and “rare” fragile sites based on frequency (Wang Y. H., “Chromatin Structure of Human Chromosomal Fragile Sites,” Cancer Lett. 232:70-78 (2006), which is hereby incorporated by reference in its entirety). In a preferred embodiment of the present invention, chromosomal integrity at or around fragile sites, FRA13B, FRA16D, FRA1K, FRA11D, FRA12B, FRA20A, FRA10C, FRA10D, FRA7I, FRA9A/9C, FRA4D, FRA5E, FRAXC, FRA18C, and FRA3B is investigated. Chromosomal integrity at or around fragile sites FRA5A, FRA8D, FRA19B and FRA6F can also be investigated.

In a preferred embodiment of the present invention, the presence of chromosomal alterations at one or more fragile sites in a genomic sample obtained from a potential premalignant condition is compared to the presence of chromosomal alterations at corresponding fragile sites in a constitutional genomic sample. As used herein, a constitutional sample is a genomic sample that is not associated with the premalignant condition. Likewise, the presence of a genomic deletion at the FRA3B sites in a genomic sample obtained from a premalignant condition is compared to the presence of a genomic deletion at the FRA3B site in a constitutional genomic sample. Preferably, the constitutional sample and the premalignant sample to be tested are obtained from the same human subject. The constitutional sample should be obtained from normal, non-premalignant tissue.

Methods and assays for determining chromosomal alterations in a genomic DNA sample are well known in the art. Such methods include but are not limited to nucleic acid amplification assays, hybridization assays, nucleic acid sequencing assays, and restriction digestions assays or any combination of the aforementioned methods. In a preferred embodiment, such assays are adapted for high through-put analysis.

A variety of nucleic acid amplification assays that are well known in the art are based on the polymerase chain reaction (PCR). General methodology for performing PCR is described in detail in U.S. Pat. Nos. 4,683,195, 4,683,202 and 4,800,159 all to Mullis et al, each of which is incorporated herein by reference in their entirety. Briefly, in PCR, two oligonucleotide primers are prepared which have sequences that are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will extend the primers along the target sequence through the addition of nucleotides. By raising and lowering the temperature of the reaction mixture, the extended primers will dissociate from the target to form reaction products, excess primers will bind to the target and to the reaction product and the process is repeated. Variations of the PCR method, including quantitative PCR methods as described herein and in U.S. Pat. Nos. 5,925,517, 6,103,476, 6,150,097, and 6,037,130 all to Tyagi, which are hereby incorporated by reference in their entirety, are also useful for carrying out the methods of the present invention.

In a preferred embodiment of the present invention, long-extension PCR is utilized for the detection of a genomic deletion In long-extension PCR the oligonucleotide primers are designed to be complementary to DNA regions upstream and downstream of the target deletion. In the absence of the deletion, the primers are located at a distance which precludes the DNA polymerase from synthesizing a PCR product. In the event of a deletion, the primers are brought in close proximity, i.e. within approximately 30 kb or less, to enable the DNA polymerase to function and product formation to occur.

The formation of an extension product using long-extension PCR, which can be detected using gel electrophoresis, fluorescent detection, or any alternative detection assay known in the art, indicates the presence of the genomic deletion. The sequence containing the deletion can in principle be detected as a very rare event in a mixed population of sequences, because only the deletion sequence is a substrate for the PCR reaction.

Because genomic deletions can vary slightly depending on the individual subject and can vary with the progression of the premalignant condition, it is preferable that a number of oligonucleotide primer sets, having overlapping complementarity for the region upstream and downstream of the deletion are utilized.

Long extension PCR using a plurality of oligonucleotide primer sets is the preferred method for detecting the genomic deletion at the FRA3B locus. In each primer sets, the first, upstream primer, has sequence complementarity to at least a portion of intron 4 of the FHIT gene, and the second, downstream primer has sequence complementarity to at least a portion of intron 5 of the FHIT gene. In normal tissue or cells in which the FRA3B deletion is not present, the primers span approximately 250 kb and the DNA polymerase will not form an extension product. However, in the event of a deletion, the primers are close enough to allow the DNA polymerase to form an extension product.

In addition to the polymerase chain reaction, alternative amplification procedures that are readily available in the art may also be employed in the methodology disclosed herein. For example, other suitable methods include ligase detection reaction (LDR) and ligase chain reaction (LCR). The ligase detection reaction process is described generally in U.S. Pat. Nos. 5,494,810, 5,830,711, and 6,054,564 to Barany et al.; Barany et al., “Cloning, Overexpression and Nucleotide Sequence of a Thermostable DNA Ligase-Encoding Gene,” Gene 109:1-11 (1991); and Barany et al., “Genetic Disease Detection and DNA Amplification Using Cloned Thermostable Ligase,” Proc. Natl. Acad. Sci. USA 88:189-193 (1991), the disclosures of which are hereby incorporated by reference in their entirety. The ligase detection reaction can use two sets of complementary oligonucleotides. This is known as the ligase chain reaction which is described in the immediately preceding references, which are hereby incorporated by reference in their entirety. Alternatively, the ligase detection reaction can involve a single cycle which is also known as the oligonucleotide ligation assay (see Landegren et al., “A Ligase-Mediated Gene Detection Technique,” Science 241:1077-80 (1988); and U.S. Pat. No. 4,988,617 to Landegren et al., which are hereby incorporated by reference in their entirety).

Alternative amplification methods that can be employed in the methods of the present invention include, but are not limited to, amplification using the RNA polymerase, Qβ Replicase, as described in WO1987006270 to Chu et al., which is hereby incorporated by reference in its entirety; isothermal amplification as described by Walker et al., “Strand Displacement Amplification—An Isothermal, In Vitro DNA Amplification Technique,” Nucleic Acids Res. 20(7):1691-96 (1992), which is hereby incorporated by reference in its entirety; strand displacement amplification (SDA); repair chain reaction (RCR); catalytic hybridization amplification (CHA) as described in WO1989009284 to Walder et al., which is hereby incorporated by reference in its entirety; nucleic acid sequence-based amplification (NASBA) as described by Deiman et al., “Characteristics and Applications of Nucleic Acid Sequence-Based Amplification (NASBA),” Mol Biotechnol. 20(2): 163-79 (2002), which is hereby incorporated by reference in its entirety; transcription-based amplification systems (TAS) described by Kwoh et al., “Transcription-Based Amplification System and Detection of Amplified Human Immunodeficiency Virus Type 1 with a Bead-Based Sandwich Hybridization Format,” PNAS 86(4):1173-7 (1989), which is hereby incorporated by reference in its entirety; rapid amplification of complementary DNA ends (RACE) as described by Frohman, “Rapid Amplification of Complementary DNA Ends for Generation of Full-Length Complementary DNAs: Thermal RACE,” Methods of Enzymology 218:340-56 (1993), which is hereby incorporated by reference in its entirety; and one-sided PCR as described by Ohara, “One-Sided Polymerase Chain Reaction: The Amplification of cDNA,” PNAS 86(15):5673-77 (1989), which is hereby incorporated by reference in its entirety.

Chromosomal alterations can also be detected using hybridization based assays or using methods which combine nucleic acid amplification with hybridization techniques. A variety of hybridization assays and detection methods are known and available in the art, including, but not limited to Southern Blots, Slot Blots, Dot Blots, and DNA microarrays. In a typical hybridization assay, an unknown nucleotide sequence (the target) is analyzed based on its affinity for another fragment with a known nucleotide sequence (the probe). If the two fragments hybridize under stringent conditions, the sequences are thought to be complementary, and the sequence of the target fragment may be inferred from the probe sequence.

Comparative genomic hybridization (CGH) is particularly useful for carrying out the methods of the present invention. Using CGH, purified genomic DNA obtained from premalignant cells and normal or control cells is labeled with different fluorochromes. The labeled DNA samples are mixed and allowed to competitively hybridize to immobilized normal DNA. The presence of chromosomal alterations, such as copy loss or gain, will be determined by differential binding of the premalignant and normal DNA to the immobilized DNA and will be detected by the differential emissions from the fluorochromes. Genomic instability can be measured using standard chromosomal-CGH methodology or microarray-based CGH, adapted for high-throughput analysis.

An array-based comparative genome hybridization (array-CGH) approach provides a robust method for carrying out genome-wide scans to find genomic variations. Genomic clones (for example, BACs), cDNAs, PCR products and oligonucleotides can all be used as array targets. The use of array-CGH with BACs is particularly popular, owing to the extensive coverage of the genome it provides, the availability of reliable mapping data and ready access to clones.

A variety of DNA microarrays, particularly those described and used in the Examples herein, are useful for the detection of chromosomal alterations and genetic variations. In addition, DNA microarrays, such as those described by Taton et al., “Scanometric DNA Array Detection with Nanoparticle Probes,” Science 289:1757-60 (2000); Lockhart et al., “Genomics, Gene Expression and DNA Arrays,” Nature 405:827-836 (2000); Gerhold et al., “DNA Chips: Promising Toys Have Become Powerful Tools,” Trends in Biochemical Sciences 24:168-73 (1999); and Wallace R W, “DNA on a Chip: Serving UP the Genome for Diagnostics and Research,” Molecular Medicine Today 3:384-89 (1997, which are hereby incorporated by reference in their entirety, are also useful for carrying out the methods of the present invention. DNA microarrays are fabricated by high-speed robotics, on glass or nylon substrates, and contain DNA fragments, i.e. probes, with known identities. The microarrays are used for matching known and unknown DNA fragments based on traditional base-pairing rules. The advantage of DNA microarrays is that one DNA chip may provide information on thousands of genes simultaneously.

The DNA microarray can be a GeneChip® (Affymetrix, Santa Clara, Calif.; see e.g., U.S. Pat. Nos. 6,045,996 to Cronin et al.; 5,925,525 to Fodor et al.; and 5,858,659 to Sapolsky et al.; each of which is herein incorporated by reference) assay. The GeneChip® technology uses miniaturized, high-density arrays of oligonucleotide probes affixed to a “chip.” The nucleic acid to be analyzed is isolated, amplified by PCR, and labeled with a fluorescent reporter group. The labeled DNA is then incubated with the array using a fluidics station. The array is inserted into the scanner, where patterns of hybridization are detected. The hybridization data are collected as light emitted from the fluorescent reporter groups already incorporated into the target, which is bound to the probe array. Since the sequence and position of each probe on the array are known, by complementarity, the identity of the target nucleic acid applied to the probe array can be determined.

Another form of the DNA microarray methodology that is useful for the present invention is the “bead array” used for the detection of polymorphisms (Illumina, San Diego, Calif.; See e.g., PCT Publications WO 99/67641 to Chee et al., and WO 00/39587 to Chee et al., each of which is herein incorporated by reference). Illumina's® BeadArray™ technology combines fiber optic bundles and beads that self-assemble into an array. The beads are coated with hundreds of thousands of copies of an oligonucleotide specific for the detection of a given SNP or mutation. Batches of beads are combined to form a pool specific to the array. To perform an assay, the BeadArray is contacted with a prepared subject sample (e.g., DNA).

Additional microarray technologies that can also be used to carry out the methods of the present invention include, but are not limited to, the NanoChip® Electronic Microarray containing electronically captured probes (Nanogen, San Diego, Calif.) (see e.g., U.S. Pat. Nos. 6,017,696 to Heller et al.; 6,068,818 to Ackley et al.; and 6,051,380 to Sosnowski et al.; each of which are herein incorporated by reference). Alternatively, the ProtoGene microarray technology, which is based upon the segregation of fluids on a flat surface (chip) by differences in surface tension (ProtoGene, Palo Alto, Calif.) can be utilized (see e.g., U.S. Pat. Nos. 6,001,311 to Brennan et al.; 5,985,551 to Brennan et al.; and 5,474,796 also to Brennan; each of which is herein incorporated by reference).

Other methods specifically developed for the rapid detection of chromosomal rearrangements and/or deletions include those described in U.S. Pat. No. 7,208,274 to Dhallen, U.S. Pat. No. 6,895,337 to Scholl et al., and U.S. Pat. No. 6,475,739 to Brunkow which are hereby incorporated by reference in their entirety.

In yet another embodiment of the present inventions, chromosomal alterations and genomic deletions can be detected using standard DNA sequencing techniques known in the art. Such methods of nucleic acid sequencing include pyrosequencing as described by Ronaghi et al., “Real-time DNA Sequencing Using Detection of Pyrophosphate Release,” Analytical Biochem. 242(1):84-89 (1996), which is hereby incorporated by reference in its entirety; the Sanger dideoxy method which utilizes enzymatic elongation procedures with chain terminating nucleotides as described in Sanger et al, “DNA Sequencing with Chain-Terminating Inhibitors,” Proc. Natl. Acad. Sci. USA 74:5463-5467 (1977) which is hereby incorporated by reference in its entirety; and the Maxam and Gilbert method which utilizes chemical reactions exhibiting specificity of reaction to generate nucleotide specific cleavages as described in Maxam et al., “Sequencing End-Labeled DNA with Base-Specific Chemical Cleavages,” Methods in Enzymology 65:499-559 (1980) which is hereby incorporated by reference in its entirety. Additionally, the methods and apparatus for sequencing nucleic acids as disclosed in WO/92/010588 to Fodor et al, which is hereby incorporated by reference in its entirety, can also be used.

Another aspect of the present invention includes a diagnostic kit which contains a detection assay for determining, in a DNA sample from a human subject, chromosomal alterations at one or more sites within one or more fragile chromosome sites. The fragile chromosome sites include FRA13B, FRA16D, FRA1K, FRA11D, FRA12B, FRA20A, FRA10C, FRA10D, FRA7I, FRA9A/9C, FRA4D, FRA5E, FRAXC, and FRA18C. Additionally, the diagnostic kit is capable of detecting a genomic deletion at fragile site FRA3B.

The high density detection assay of the diagnostic kit is preferably a high-throughput assay and can include, but is not limited to, any of the nucleic acid amplification, hybridization, sequencing assays, or combination thereof as described above. In one embodiment, the high density detection assay comprises one or more oligonucleotide primer sets wherein each primer set is designed to detect a chromosomal alteration in one or more specific fragile chromosome site. The diagnostic kit may further contain reagents for carrying out an amplification assay (e.g. DNA polymerase, ligase, buffers, dNTPs, etc). The diagnostic kit of the present invention may further include a solid support (e.g. an array) containing oligonucleotide probes that are useful for detecting and distinguishing chromosomal alterations at one or more specific fragile chromosome site.

In a preferred embodiment of the present invention, the diagnostic kit contains reagents for carrying out long extension PCR. Such reagents include one or more oligonucleotide primer sets, where each primer set is designed to be complementary to DNA regions upstream and downstream of the target fragile chromosome deletion. Preferably, a plurality of oligonucleotide primer sets are included in the kit, each primer set having overlapping complementarity to regions upstream and downstream of the target deletion area. The oligonucleotide primers may further contain a label, e.g. fluorescent label, to facilitate detection of the PCR product. The kit of the present invention may also include a DNA polymerase enzyme (e.g. Taq polymerase), PCR buffer, dNTPs, and one or more control DNA samples.

In addition to diagnosing a premalignant condition, the methods of the present invention can further be utilized to establish a prognosis for the premalignant condition diagnosed. Prognosis as described herein includes determining the probable course and outcome of the premalignant condition and can include determining the risk of developing cancer. A prognosis of a premalignant condition can be based on the presence of chromosomal alterations at one or more of the fragile chromosome sites discussed supra. Likewise, a prognosis of a premalignant condition may be based on the presence or extent of a genomic deletion at the FRA3B fragile site.

The methods of the present invention can also be utilized to establish a therapeutic regimen for the subject diagnosed with the premalignant condition. A therapeutic regimen as used herein includes developing a treatment plan that is specifically tailored for the subject having the premalignant condition based on the presence of chromosomal alterations. The therapeutic regimen for the subject can also be adjusted based on changes in the chromosomal alterations over the progression of the condition. One or more genomic DNA samples can be collected from the subject at different timepoints during the progression of the premalignant condition and the chromosomal alterations at one or more of the fragile chromosome sites is monitored. Based on changes in the size or location of the chromosomal alterations, the therapeutic regimen may be adjusted accordingly.

In a preferred embodiment, the therapeutic regimen for a human subject having a premalignant or malignant condition, is based on the presence of a genomic deletion at the FRA3B fragile site. The size and extent of the FRA3B deletion can be monitored at various timepoints during the progression of the premalignant condition and changes in the size or extent of the deletion can be useful for determining an optimal therapeutic regimen or adjusting the therapeutic regimen for a human subject having a premalignant or malignant condition.

### EXAMPLES

### Example 1

### Samples and DNA Extraction

Patients were members of the Seattle Barrett's Esophagus Surveillance Program prospective surveillance cohort and were managed according to the protocol previously described (Reid et al., “Flow-Cytometric and Histological Progression to Malignancy in Barrett's Esophagus: Prospective Endoscopic Surveillance of a Cohort.” Gastroenterology 102:1212-1219 (1992), which is hereby incorporated by reference in its entirety). Endoscopic mucosal biopsies were obtained as described previously and were graded for the presence or absence of metaplasia and dysplasia according to previously documented histological criteria (Reid et al., “Flow-Cytometric and Histological Progression to Malignancy in Barrett's Esophagus: Prospective Endoscopic Surveillance of a Cohort,” Gastroenterology 102:1212-1219 (1992), which is hereby incorporated by reference in its entirety). Three patients were selected who were observed to progress through three distinct stages of molecular evolution in BE during their surveillance: (1) CDKN2A^{LOH}, (2) CDKN2A^{LOH}/TP53^{LOH}, or (3) CDKN2A^{LOH}/TP53^{LOH} with aneuploidy, based on genotyping and flow cytometric procedures previously described (Galipeau et al., “Clonal Expansion and Loss of Heterozygosity at Chromosomes 9p and 17p in Premalignant Esophageal (Barrett's) Tissue,” J Natl Cancer Inst 91:2087-2095 (1999); Rabinovitch et al., “Predictors of Progression in Barrett's Esophagus III: Baseline Flow Cytometric Variables,” Am J Gastroenterol 96:3071-3083 (2001), which are hereby incorporated by reference in their entirety). Endoscopies from the same individual were selected within 5 cm region of the BE segment. Genomic DNA was extracted from Ki-67 positive flow-sorted nuclei using the Puregene DNA isolation kit as previously described (Paulson et al., “Loss of Heterozygosity Analysis Using Whole Genome Amplification, Cell Sorting, and Fluorescence-Based PCR,” Genome Res 9:482-491 (1999), which is hereby incorporated by reference in its entirety). For each patient, lymphocyte constitutional DNA was isolated from buffy coat preparations using the Qiagen DNeasy kit as per manufacturer's instructions. The histology and molecular characteristics of each biopsy are summarized in Table 1. The Seattle Barrett's Esophagus Study has been approved by the Human Subjects Divisions of the University of Washington and the Fred Hutchinson Cancer Research Center (FHCRC) continuously from 1983 to the present.

TABLE 1
Clinical Data for Patient Group

| | Sample | | | | | | | |
|---|---|---|---|---|---|---|---|---|
| Patient | stage | Months^{a} | Fraction | CDKN2A^{LOH} | TP53^{LOH} | Ploidy | Histology^{b} | Cancer |
| 1 | A | 0 | G1 | Yes | No | 2N | Indefinite | |
| | B | 6 | G1 | Yes | Yes | 2N | High-Grade | |
| | C | 33 | Aneuploid | Yes | Yes | 2.86N | High-Grade^{b} | No |
| 2 | A | 0 | G1 | Yes | No | 2N | Metaplasia | |
| | | | | | | | indefinite | |
| | B | 36 | G1 | Yes | Yes | 2N | High-Grade | |
| | C | 36 | Aneuploid | Yes | Yes | 3.82N | High-Grade | Yes^{c} |
| 3 | A | 0 | G1 | Yes | No | 2N | Indefinite | |
| | B | 10 | G1 | Yes | Yes | 2N | Low-Grade | |
| | C | 22 | Aneuploid | Yes | Yes | 2.97N | High-Grade | Yes (focal)^{d} |
| 4 | A | 0 | G1 | Yes | No | 2N | Metaplasia | No |
| 5 | A | 0 | Gl | Yes | No | 2N | Indefinite | No |
| 6 | A | 0 | Gl | Yes | No | 2N | Low-Grade | No |

^{a}Time in months between endoscopic sampling; the blood sample was taken at the same time as the initial BE diploid CDKN2A^{LOH} sample.
^{b}Histology is derived from the level at which the biopsy is taken, except in case 1C in which only the maximum grade diagnosis at the endoscopy was available.
^{c}Cancer was present at the same time as sample C was taken, but at a site 3 cm distant.
^{d}One biopsy of 9 at this level showed focal cancer and no cancer was found in the resected specimen.

### Example 2

### BAC Arrays

Genomic DNA (10 ng) was digested with Dpn I and processed by the Fred Hutchison Cancer Research Center Microarray Facility for analysis on BAC arrays as previously described (Loo et al., “Array Comparative Genomic Hybridization Analysis of Genomic Alterations in Breast Cancer Subtypes,” Cancer Res 64:8541-8549 (2004), which is hereby incorporated by reference in its entirety). DNA was hybridized along with a labeled genomic male reference sample (Promega). Scanned images were analyzed using GenePix 6.0 and analyzed for copy number changes using normalization and mapping programs in R developed by Douglas Grove, Fred Hutchison Cancer Research Center (Loo et al., “Array Comparative Genomic Hybridization Analysis of Genomic Alterations in Breast Cancer Subtypes.” Cancer Res 64:8541-8549 (2004), which is hereby incorporated by reference in its entirety).

### Example 3

### SNP Array CGH Analysis

Genomic DNA was processed for array hybridization according to the protocol for Affymetrix GeneChip® mapping 100K arrays (Affymetrix, Inc.). DNA samples were hybridized to one of the two 50K (Hind and Ma) arrays that comprised this set, and scanned according to manufacturer's instructions. CEL images were analyzed using Affymetrix GCOS version 1.4 and GTYPE software version 4.0 with heterozygosity rate set at 0.3 and P value set at default 0.25 to generate genotype calls. P value criteria were set at 0.05 for the SNP's. Call rates for the arrays averaged 94.10%±3.2%. Copy number data were generated for each array by the Affymetrix Chromosome Copy Number Tool (CNAT) v.2.0 (Huang et al., “Whole Genome DNA Copy Number Changes Identified by High Density Oligontocleotide Arrays,” Hum Genomics 1:287-299 (2004), which is hereby incorporated by reference in its entirety); single point analysis (SPA) values were used for filtering and for copy number calculations, Plots reflect 0.5 Mb smoothed data for the log₂ ratio of experimental versus lymphocyte reference control.

Regions of significant gain or loss in copy number were assessed using the Cluster Along Chromosomes (CLAC) program (Wang et al., “A Method For Calling Gains and Losses in Array CGH Data,” Biostatistics 6:45-58 (2005), which is hereby incorporated by reference in its entirety). For copy number assessment, log₂ ratios of SPA values for chromosomes 1-22 and X were used, the false discovery rate (FDR) was set at 0.01 level, a 5 SNP moving average was specified, and genome centering was not applied. Significant copy number alterations were further filtered for changes above or below 20% (i.e., gains, log₂ ratio >0.263; losses, log₂ ratio <−0.321). For size of copy gain/loss, nucleotide positional difference (bp) was estimated using the positions of the most 5′ and 3′ SNP's within continuous regions of gain/loss. The Kruskal-Wallis test was applied to determine whether significant differences in the mean length of regions of copy gain, copy loss, or LOH exist within patients at each of the three different molecular stages. The Cochran-Mantel-Haenszel test was used to evaluate whether there was a significant trend of incidence of copy gain, copy loss, or LOH over the three molecular stages adjusted for each patient.

Genotype calls were filtered first with genotype call P value (P≦0.05) then by z-score threshold for signal intensity. The MPAM mapping algorithm was applied to raw probe intensity data exported from GTYPE v 4.0 to generate RAS1 and RAS2 scores as outlined by the Affymetrix GTYPE user guide. Z scores were calculated as a measurement of distance to the ordinate of the median RAS1 and RAS2 scores for each SNP. To filter the data, genotype calls were excluded if the intensities fell outside the mean z-score ±3 SD per genotype cluster per array. Typically, 1-2% of all calls were excluded using z-score filtering criteria. Regions of contiguous LOH and copy number change were independently confirmed using dChip2004, (Lin et al., “dChipSNP: Significance curve and clustering of SNP-Array-Based Loss-of-Heterozygosity Data,” Bioinformatics 20:1233-1240 (2004), which is hereby incorporated by reference in its entirety). Absolute values for delta Z scores were run in the CLAC program to identify regions of LOH. Correlations of copy number and genotype were generated using CLAC output for copy number and genotypic data.

For generation of consensus plots, region mean output from CLAC analysis of paired CDKN2A^{LOH}-only metaplastic and reference samples from the combined Hind and Xba arrays were merged into a single file. Copy number gains or losses were inferred to cover intervening SNP's from the other 50K chip. Consensus plots were generated using the CLAC program with the assumption that zero or ND values represented no alteration.

### Example 4

### Results

To evaluate genomic instability, including both copy number alterations and allelic changes, during neoplastic progression of BE, three patients were selected, each with endoscopic biopsies that over a two to three year time course showed sequential advancement through the three distinct molecular stages previously demonstrated to be important in the neoplastic evolution in this condition (Galipeau et al., “Clonal Expansion and Loss of Heterozygosity at Chromosomes 9p and 17p in Premalignant Esophageal (Barrett's) Tissue,” J Natl Cancer Inst 91:2087-2095 (1999); Rabinovitch et al., “Predictors of Progression in Barrett's Esophagus III: Baseline Flow Cytometric Variables,” Am J Gastroenterot 96:3071-3083 (2001); Wong et al., “p16(INK4a) Lesions Are Common, Early Abnormalities That Undergo Clonal Expansion in Barrett's Metaplastic Epithelium,” Cancer Res 61:8284-8289 (2001); Maley et al., “The Combination of Genetic Instability and Clonal Expansion Predicts Progression to Esophageal Adenocarcinoma,” Cancer Res 64:7629-7633 (2004), which are hereby incorporated by reference in their entirety). These molecular stages are: (1) CDKN2A^{LOH} alone, (2) CDKN2A^{LOH}/TP53^{LOH}, and (3) CDKN2A^{LOH}/TP53^{LOH} with aneuploidy. The histology of biopsies with CDKN2A^{LOH} alone was metaplasia, indefinite or low-grade dysplasia, whereas the histology when TP53^{LOH} was present was low- or high-grade dysplasia (Table 1). For comparison, endoscopic biopsies were also selected from three BE patients that had only CDKN2A^{LOH} (no evidence of aneuploidy or TP53^{LOH}) over the duration of their surveillance (at least 4 examinations over a mean follow-up 12 years). DNA extracted from flow-sorted Barrett's epithelium and lyimphocyte controls were run on single Affymetrix GeneChip 50K mapping arrays of the 100K set.

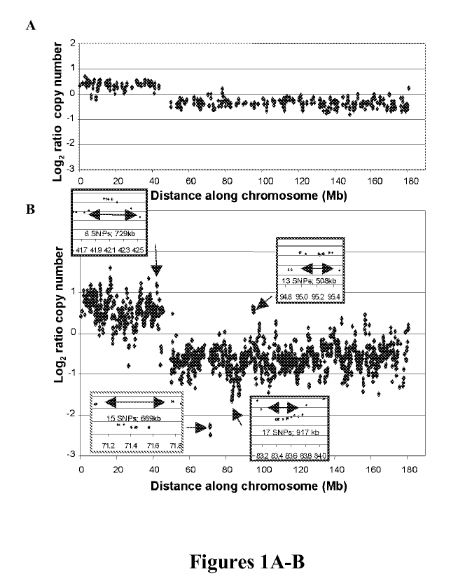

FIGS. 1A-B show representative chromosome plots, illustrating the difference in copy number estimates derived from a 5K BAC array (FIG. 1A) versus an Affymetrix 50K Hind array (FIG. 1B). Typically, a good correlation between the two platforms for large changes (>1 Mb) was observed. For example, gain of the p-arm and loss of the q-arm of chromosome 5 as shown were clearly visible. However, as seen in this example, copy alterations of smaller extent were evident only using the higher density SNP arrays. At higher resolution, it can be seen that many regions of loss and gain comprised ten or more SNP's, sometimes spanning only a few hundred kilobases (see FIG. 1B insets). These small events are not visible in BAC arrays in which the probe size is large (average 150 kb) and the density is low. In addition, chromosomal breakpoints could be estimated with much greater accuracy with the small median inter-marker distance of the high density arrays. It is of note that homozygous deletions were also observed (see chromosome 5 at 70 Mb) using the high density array chips; the accurate assessment of both copy number and allelic ratios are facilitated by the enrichment of epithelial cells from the endoscopic biopsies by flow-sorting.

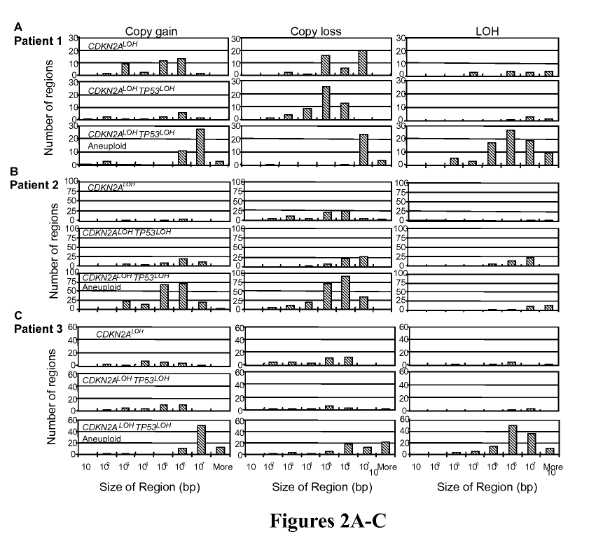

To further illustrate the pattern of evolutionary dynamics of the regions of gain, loss, or LOH over time, histograms were plotted of the frequency of copy gain, copy loss, and LOH events versus the size of the chromosomal regions involved (FIG. 2). Small sized copy loss and gain events (<1 Mb) that were made visible in SNP arrays (FIG. 1B) were common in early stage (CDKN2A^{LOH} only) disease, Both the number of events, including copy number loss, copy number gain, and LOH, and the size of copy loss and copy gain events increased with advancing molecular stage (all P <001). Regions of LOH increased in number (P <0.001) but not size (P >0.57) with advancing stage.

Table 2 shows the quantitation of LOH calls, which generally increased with stage of neoplastic progression. In metaplastic CDKN2A^{LOH} only samples (designated as sample type A in each patient) an average of 5.6% of informative SNPs showed LOH, increasing to an average of 19.9% in the aneuploid samples (sample type C) (P<0.0001). To further address the mechanisms underlying the genomic instability in the BE samples, results from LOH and copy number analyses were combined to distinguish between regions of LOH associated with copy loss, copy gain, or no copy number change (Table 3). LOH with copy loss would reflect acquired hemizygosity. LOH with copy number gain would reflect allelic imbalance, resulting from an amplification event or copy loss following or followed by aneuploidy or tetraploidy. Copy neutral events presumably result from a recombinational event, such as mitotic recombination. Very few instances of LOH associated with copy number gain (<10% in all samples) were observed. However, there was a significant trend of an increasing proportion of LOH events associated with copy loss with increase in molecular stage (P <0.003), although in Patients 2 and 3, the large increase in this proportion from CDKN2A^{LOH} only to CDKN2A^{LOH}/TP53^{LOH} samples did not further increase in the aneuploid samples.

TABLE 2
Summary of LOH Calls Detected in BE Samples

| Patient | Molecular stage | No. of LOH No. of inform % LOH (%) | | |
|---|---|---|---|---|
| 1 | A. Diploid CDKN2A^{LOH} | 1,035 | 9,367 | 9.95 |
| | B. Diploid CDKN2A^{LOH}TP53^{LOH} | 324 | 12,407 | 2.54 |
| | C. Aneuploid CDKN2A^{LOH}TP53^{LOH} | 1,790 | 8,339 | 17.67 |
| 2 | A. Diploid CDKN2A^{LOH} | 73 | 4,794 | 1.50 |
| | B. Diploid CDKN2A^{LOH}TP53^{LOH} | 309 | 4,216 | 6.83 |
| | C. Aneuploid CDKN2A^{LOH}TP53^{LOH} | 914 | 3,674 | 19.92 |
| 3 | A. Diploid CDKN2A^{LOH} | 189 | 9,868 | 1.88 |
| | B. Diploid CDKN2A^{LOH}TP53^{LOH} | 77 | 9,000 | 0.85 |
| | C. Aneuploid CDKN2A^{LOH}TP53^{LOH} | 1,722 | 6,045 | 22.17 |
| 4 | A. Diploid CDKN2A^{LOH} | 364 | 13,346 | 2.65 |
| 5 | A. Diploid CDKN2A^{LOH} | 405 | 13,353 | 2.94 |
| 6 | A. Diploid CDKN2A^{LOH} | 1,860 | 10,870 | 14.61 |

SNPs with heterozygous (informative, inform) calls in the constitutional sample and homozygous calls in the BE sample were designated as LOH calls. % LOH is number of LOH calls relative to the total number of informative calls.

TABLE 3
Association of LOH with Copy Change (Loss or Gain) and Molecular Stage

| | | | No. of | % LOH + | No. of | % LOH + |
|---|---|---|---|---|---|---|
| | | Regions | LOH + | copy gain^{a} | LOH + | copy loss^{a} |
| Patient | Molecular stage | of LOH | copy gain^{a} | (%) | copy loss^{a} | (%) |
| 1 | A. Diploid CDKN2A^{LOH} | 14 | 1 | 7.1 | 1 | 7.1 |
| | B. Diploid | 6 | 0 | 0.0 | 0 | 0.0 |
| | CDKN2A^{LOH}TP53^{LOH} | | | | | |
| | C. Aneuploid | 80 | 2 | 2.5 | 17 | 21.2 |
| | CDKN2A^{LOH}TP53^{LOH} | | | | | |
| 2 | A. Diploid CDKN2A^{LOH} | 3 | 0 | 0.0 | 0 | 0.0 |
| | B. Diploid | 46 | 0 | 0.0 | 25 | 54.3 |
| | CDKN2A^{LOH}TP53^{LOH} | | | | | |
| | C. Aneuploid | 28 | 0 | 0.0 | 19 | 67.8 |
| | CDKN2A^{LOH}TP53^{LOH} | | | | | |
| 3 | A. Diploid CDKN2A^{LOH} | 7 | 0 | 0.0 | 1 | 14.2 |
| | B. Diploid | 6 | 0 | 0.0 | 4 | 66.6 |
| | CDKN2A^{LOH}TP53^{LOH} | | | | | |
| | C. Aneuploid | 120 | 3 | 2.5 | 77 | 64.1 |
| | CDKN2A^{LOH}TP53^{LOH} | | | | | |

Values shown are the total number of genomic regions containing two or more consecutive LOH calls. Regions with both LOH calls and copy gain or loss were considered to have copy gain or loss associated with LOH.
^{a}copy number gain or loss as detected by the CLAC program.

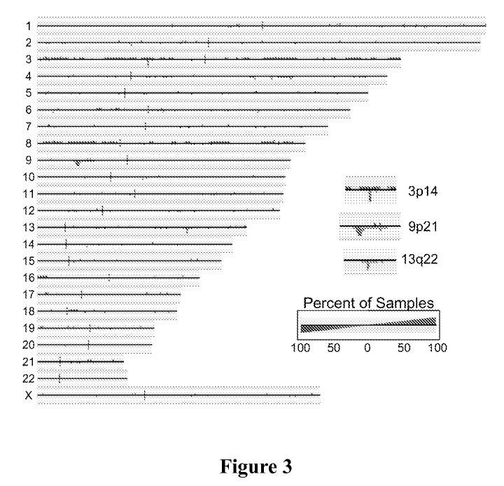

To investigate whether conserved regions of copy number gain or loss could be identified among all BE patients examined, a consensus plot was generated for all six of the CDKNA2A^{LOH}-only metaplastic samples (FIG. 3). Most regions of copy number gain or loss were detected in only one of the six samples, suggesting that they were random events or were not significantly associated with a common mechanism of neoplastic progression of Barrett's esophagus. There were no regions of copy gain that were conserved in more than half of the samples. However, three regions of loss were detected in the majority of samples tested. Copy number loss was detected in five of six samples at both the CDKN2A locus on chromosome band 9p21 and at the FHIT locus on chromosome band 3p14. An additional region of loss on chromosome band 13q22 was detected in four of six samples. Although copy loss at the CDKN2A locus is consistent with previous reports on the importance of this locus in early BE neoplastic progression (Galipeau et al., “Clonal Expansion and Loss of Heterozygosity at Chromosomes 9p and 17p in Premalignant Esophageal (Barrett's) Tissue,” J Natl Cancer Inst 91:2087-2095 (1999); Wong et al., “p16(INK4a) Lesions Are Common, Early Abnormalities That Undergo Clonal Expansion in Barrett's Metaplastic Epithelium,” Cancer Res 61:8284-8289 (2001); Maley et al., “Selectively Advantageous Mutations and Hitchhikers in Neoplasms: p16 Lesions Are Selected in Barrett's Esophagus,” Cancer Res 64:3414-3427 (2004b), which are hereby incorporated by reference in their entirety), the FHIT and 13q22 loci are the locations of fragile sites (FRA3B and FRA13B, respectively). There were no other regions of loss seen more than once among the six samples.

A search for conserved regions of LOU across samples also identified the CDKN2A locus on chromosome arm 9p in all six of the earliest stage CDKN2A^{LOH} only samples, consistent with previous analysis by MSI allelotyping (Gonzalez et al., “Mutation Analysis of the p53, APC, and p16 Genes in the Barrett's Oesophagus, Dysplasia, and Adenocarcinoma,” J Clin Pathol 50:212-217 (1997); Galipeau et al., “Clonal Expansion and Loss of Heterozygosity at Chromosomes 9p and 17p in Premalignant Esophageal (Barrett's) Tissue,” J Natl Cancer Inst 91:2087-2095 (1999); Wong et al., “p16(INK4a) Lesions Are Common, Early Abnormalities That Undergo Clonial Expansion in Barrett's Metaplastic Epithelium,” Cancer Res 61:8284-8289 (2001), which are hereby incorporated by reference in their entirety). Also consistent with the MSI allelotyping (Galipeau et al., “Clonal Expansion and Loss of Heterozygosity at Chromosomes 9p and 17p in Premalignant Esophageal (Barrett's) Tissue,” J Natl Cancer Inst 91:2087-2095 (1999), which is hereby incorporated by reference in its entirety), no LOH was observed at the TP53 locus in any of these CDKN2A^{LOH}-only samples. LOH at the FHIT locus was seen in four of six CDKN2A^{LOH}-only samples. LOH at several loci previously suggested to be involved in BE progression, such as DCC, RBI, and APC was also checked; however, LOH at these loci was each detected in only one of six samples. No LOH was detected at the SMAD4 locus (Dolan et al., “TTP53 Mutations in Malignant and Premalignant Barrett's Esophagus,” Dis Esophagus 16:83-89 (2003); Sanz-Ortega et al., “3p21, 5q21, 9p21 and 17p13.1 Allelic Deletions Are Potential Markers of Individuals With a High Risk of Developing Adenocarcinoma in Barrett's Epithelium Without Dysplasia,” Hepatogastroenterology 50:404-407 (2003); Hage et al., “Genomic Analysis of Barrett's Esophagus After Ablative Therapy: Persistence of Genetic Alterations at Tumor Suppressor Loci,” Int J Cancer 118:155-160 (2006), which are hereby incorporated by reference in their entirety). Within the three years of follow-up and advancing molecular stage, additional regions of LOH appeared on chromosome arms 5q, 9p, and 17p (% SNP's, with LOH averaging 98.3%, 69.6%, and 96%, respectively, in the three aneuploid samples). Increasing levels of LOH were also observed on chromosome arms 1q, 2q, 5p, 7q, 8p, 8q, 10q, 11q, 12p, 12q, 17q, 15q, 20p, and chromosome 21 during neoplastic progression, but with a lesser degree of conservation.

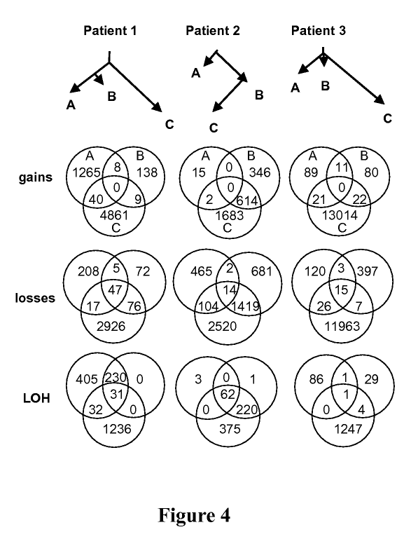

To examine the utility of SNP arrays to discern relationships of clonal evolution between samples, the overlap of individual SNP's with copy loss, copy gain, and LOH between samples at each of the three molecular stages of progression was compared (See FIG. 4). A screen for conservation among significant SNP calls using the CLAC program with additional filtering as described supra was performed. Shared numbers of SNP's with copy number gains (top row), losses (2nd row), or LOH (3rd row) were plotted as Venn diagrams (FIG. 4). Since endoscopies for each patient were controlled for approximately the same spatial location within the BE segment, several possible scenarios were assumed. If all samples are highly related and derived from a common progenitor, then many events should be shared among all three samples, located in the center of the Venn diagram; there are relatively few SNPs in this category. If a BE segment was affected by a selective sweep (Maley et al., “Selectively Advantageous Mutations and Hitchhikers in Neoplasms: p16 Lesions Are Selected in Barrett's Esophagus,” Cancer Res 64:3414-3427 (2004b), which is hereby incorporated by reference in its entirety), then a novel clonal population could be observed over time; the aneuploid population in Patient 3 fits this paradigm, Analysis of Patient 2 demonstrated clearly that the aneuploid sample was closely derived from the CDKN2A^{LOH}/TP53^{LOH} sample; while most of the genetic alterations in these populations are not present in the CDKNA2A^{LOH} only sample, some overlap in copy loss and LOH events does suggest a common ancestor. Patient 1 shows a combination of the cases outlined above; i.e., conserved gain and LOH events between the first two samples, but also conserved losses between the second and third samples.

### Discussion of Examples 1-4

This is the first demonstration of using high density SNP arrays for a longitudinal investigation of genomic instability occurring during neoplastic progression of a pre-malignant disease through specific molecular stages. The genomic instability in BE patients during neoplastic progression was compared using specific molecular alterations, which in combination have been demonstrated to increase the relative risk of developing cancer. The use of high density arrays allowed the discrimination of significant numbers of small copy number alterations (<1 Mb) on many chromosomes in the earliest metaplastic samples (FIG. 1B). By comparison, copy number changes of this smaller size are undetectable by BAC arrays or conventional CGH (FIG. 1A). For example, previous reports showed that genomic instability using conventional CGH was detectable only in late stage BE (samples that had progressed past low-grade dysplasia) (Croft et al. “Analysis of the Premalignant Stages of Barren's Oesophagus Through to Adenocarcinoma by Comparative Genomic Hybridization,” Eur J Gastroenterol Hepatol 14:1179-1186 (2002), which is hereby incorporated by reference in its entirety).

The detection of these small events also enabled discrimination of the trends of increasing numbers and size of copy loss, copy gain, and LOH events during neoplastic progression (Table 2; FIG. 2). The ability to detect LOH and copy change on the same array also allows inter-correlation of these two categories of events. LOH appears to occur independently of copy changes in the earliest CDKN2A^{LOH} only samples (Table 3), and this suggests that recombinational mechanisms of genetic alteration may predominate in these early changes. This would also be consistent with the proposed role for ROS and oxidative damage in promoting BE (Sihvo et al., “Oxidative Stress Has a Role in Malignant Transformation in Barrett's Oesophagus,” Int J Cancer 102:551-555 (2002); Jenkins et al., “Deoxyclolic Acid (DCA) at Neutral and Acid pH, is Genotoxic to Oesophageal Cells Through the Induction of ROS: The Potential Role of Antioxidants in Barrett's Oesophagtis,” Carcinogenesis 28:136-142 (2007); Payne et al., “Deoxycholate Induces Mitochondrial Oxidative Stress and Activates NF-κB Through Multiple Mechanisms in HCT-116 Colon Epithelial Cells,” Carcinogenesis 28:215-222 (2007), which are hereby incorporated by reference in their entirety), as small interstitial LOH events have previously been found to be associated with oxidative damage to DNA (Turker et al., “A Novel Signature Mutation for Oxidative Damage Resembles a Mutational Pattern Found Commonly in Human Cancers,” Cancer Res 59:1837-1839 (1999), which is hereby incorporated by reference in its entirety). With advancing molecular stage, the size and number of LOH events increases, and a greater proportion of LOH events are associated with copy loss. This suggests a shift in the underlying mechanism(s) of instability to one that seems likely to include mitotic instability and chromosomal rearrangements. In agreement with these findings, a recent comparison of fluorescence in situ hybridization (FISH) and LOH at chromosome arm 17p (TP53 locus) found that LOH developed with copy loss, no copy change, or duplication followed by loss in approximately equal proportions in biopsies, but with more of the aneuploid than non-aneuploid TP53^{LOH} (specimens showing copy changes (Wongsurawat et al., “Genetic Mechanisms of TP53 Loss of Heterozygosity in Barrett's Esophagus: Implications for Biomarker Validation,” Cancer Epidemiol Biomark Prev 15:509-516 (2006), which is hereby incorporated by reference in its entirety). Thus, this high density array data suggests a model in which fundamental shifts in the mechanisms of genomic instability occur in early versus late disease. In the most advanced stage of premalignant BE (i.e., aneuploidy), at most a 65% correlation between regions of LOU and copy loss was observed, which was lower than the 90% correlation of MSI and copy number reported by Yano et al., “Accuracy of An Array Comparative Genomic Hybridization (CGH) Technique in Detecting DNA Copy Number Aberrations: Comparison With Conventional CGH and Loss of Heterozygosity Analysis in Prostate Cancer,” Cancer Genet Cytogenet 150:122-127 (2004), which is hereby incorporated by reference in its entirety, likely because of the inclusion of many small regions of LOH in this data. These results are more consistent with a study performed on Affymetrix 10K SNP arrays, which demonstrated progressive increases in select regions of LOH in premalignant and malignant oral epithelial cell lines (Zhou et al., “Concurrent Analysis of Loss of Heterozygosity (LOH) and Copy Number Abnormality (CNA) for Oral Premalignancy Progression Using the Affymetrix 10K SNP Mapping Array,” Hum Genet 115:327-330 (2004), which is hereby incorporated by reference in its entirety). Finally, these results clearly support the concept of a mutator phenotype (Loeb L A, “Mutator Phenotype May Be Required for Multistage Carcinogenesis,” Cancer Res 51:3075-3079 (1991), which is hereby incorporated by reference in its entirety) in premalignant tissue; the average rate of 19.9% LOH among informative SNPs in the most advanced samples indicates a tremendous mutational burden, which was also observed to a lesser extent in a subset of the earlier CDKN2A^{LOH} only samples (Table 2, FIG. 2).

In testing for conserved alterations among the early CDKN2A^{LOH} only samples, it was surprising to find only three regions of conserved copy number loss, located within FHIT/FRA3B (in chromosome band 3p14). CDKN2A (in chromosome band 9p21), and FRA13B (in chromosome band 13q22). Losses at both the FHIT and CDKN2A loci have been previously described in studies of Barrett's esophagus (Lisitsyn et al., “Comparative Genomic Analysis of Tumors: Detection of DNA Losses and Amplification,” Proc Natl Acad Sci USA 92:151-155 (1995); Michael et al., “Frequent Deletions of FHIT and FRA3B in Barrett's Metaplasia and Esophageal Adenocarcinomas,” Oncogene 15:1653-1659 (1997); Galipeau et al., “Clonal Expansion and Loss of Heterozygosity at Chromosomes 9p and 17p in Premalignant Esophageal (Barrett's) Tissue,” J Natl Cancer Inst 91:2087-2095 (1999); Albrecht et al., “Array-Based Comparative Genomic Hybridization for the Detection of DNA Sequence Copy Number Changes in Barrett's Adenocarcinoma,” J Pathol 203:780-788 (2004), which are hereby incorporated by reference in their entirety), although the high frequency of deletions at these fragile sites (83% at FRA3B, 67% at FRA131B) in the early (CDKN2A LOH only) BE biopsies was surprising. Fragile sites are hypothesized to possess chromatin structures that are difficult for the DNA replication apparatus to traverse, and deletion may be a consequence of stalling at these sites after DNA damage (Wang Y H, “Chromatin Structure of Human Chromosomal Fragile Sites,” Cancer Lett 232:70-78 (2006), which is hereby incorporated by reference in its entirety). Deletion at FRA3B and FRA13B may thus be signatures of the genotoxic stress associated with chronic reflux and inflammation in BE.

A high rate of clonal dynamics occurs within a segment of Barrett's esophagus as additional molecular alterations are acquired. Within a 3-year time period, large numbers of chromosomal copy number and allelic changes can be acquired, sustained, and lost (FIG. 4). The majority of SNPs with LOH were not maintained in all three samples from the same patient, perhaps evidence of multiple clonal sweeps across the BE segment (Maley et al., “Selectively Advantageous Mutations and Hitchhikers in Neoplasms: p16 Lesions Are Selected in Barrett's Esophagus,” Cancer Res 64:3414-3427 (2004b); Maley et al., “Genetic Clonal Diversity Predicts Progression to Esophageal Adenocarcinoma,” Nat Genet 38:468-473 (2006), which are hereby incorporated by reference in their entirety). This study of three individuals illustrates three possible clonal pathways (FIG. 4). Of note, the second and third samples from Patient 2 originated from a single biopsy (flow sorted into diploid and aneuploid populations) and shared many events; nonetheless, many additional LOH events, copy number gains, and copy number losses exclusively within the aneuploid sample were observed.

Using high density array technology, a small amount of clinical material can be used to evaluate many loci simultaneously. These results demonstrate that high resolution SNP arrays can provide genome-wide measurements of copy number change and LOH to elucidate mechanisms of genomic instability and clonal relationships during neoplastic progression of a premalignant tissue; specifically it demonstrates the changing character of increasing genomic instability during neoplastic progression in BE. While this study involved only a small number of patients, the technology enabled novel insights into neoplastic progression to be made. This demonstrates the promise of this approach and the merit of extending this method to other premalignant diseases. It also shows the potential of high density SNP arrays to identify new candidate biomarkers and measures of clonal diversity, both of which can be of use in patient management and assessment of cancer risk.

### Example 5

### Patient Population and Sampling

All patients included in this study were participants in the Seattle Barrett's Esophagus Surveillance Program and were evaluated as previously described (Reid et al., “Predictors of Progression in Barrett's Esophagus II: Baseline 17p (p53) Loss of Heterozygosity Identifies a Patient Subset At Increased Risk For Neoplastic Progression,” Am J Gastroenterol 96:2839-48 (2001), which is hereby incorporated by reference in its entirety). The endoscopic biopsies were analyzed for molecular alterations at chromosome arm 9p (p16 locus), chromosome arm 17p (p53 locus), and aneuploidy as previously described (Reid et al., “Flow-Cytometric and Histological Progression to Malignancy in Barrett's Esophagus: Prospective Endoscopic Surveillance of a Cohort,” Gastroenterology 102:1212-9 (1992), which is hereby incorporated by reference in its entirety). Samples from twenty patients were analyzed by PCR and pyrosequencing, in which 1 to 6 biopsies (separated by a minimum of 2 cm longitudinally in the BE segment) were studied from each patient; multiple biopsies had the same histologic and molecular findings. Histologic diagnosis was established by previously published criteria (Reid et al., “Flow-Cytometric and Histological Progression to Malignancy in Barrett's Esophagus: Prospective Endoscopic Surveillance of a Cohort.” Gastroenterology 102: 1212-9 (1992), which is hereby incorporated by reference in its entirety). None of these patients overlapped with those examined by array CGH as described in Examples 1-4 above. Cells from these biopsies were purified by sorting, as described below, Twenty patients with early molecular stage BE, chromosome arm 9p^{LOH}, but without chromosome arm 17p^{LOH} or aneuploidy (Table 4) in which cells from biopsies were also purified by sorting. The Seattle Barrett's Esophagus Study has been approved by the Human Subjects Divisions of the University of Washington and/or the Fred Hutchinson Cancer Research Center (FHCRC) continuously from 1983 to the present,

TABLE 4
Biopsy characteristics for samples analyzed by SNP array-CGH.

| | | | Molecular | | | | | | | |
|---|---|---|---|---|---|---|---|---|---|---|
| | | | Characterization | | | Diagnoses | | | | |
| | | | 9p | 17p | | # | # | # | # | # |
| | Group | n | LOH* | LOH* | Aneuploidy* | Meta | Indef | LGD | HGD | Cancer |
| A | Early sorted | 21 | 8 | 0 | 1** | 7 | 10 | 3 | 0 | 0 |

*Molecular characterization was not available for all samples-numbers reflect only those cases in which data was available.
**Aneuploidy was detected in another biopsy within the same patient. For diagnoses, the maximum histologic diagnosis at any biopsy site within 1 cm of the biopsy sampled for DNA is shown. Biopsy diagnoses included Meta (Metaplasia without dysplastic changes); Indef (Indefinite for dysplasia); LGD (low-grade dysplasia), HGD (high-grade dysplasia), and Cancer.

### Example 6

### Illumina SNP Genotyping

In total, 20 paired constitutional (LES or gastric) and endoscopic or surgical BE samples were analyzed by Illumina's Infinium™ assay on high density SNP genotyping BeadChips per manufacturer's instructions. In brief, genomic DNA was extracted from Ki67-positive flow sorted epithelium samples as described (Galipeau et al., “Clonal Expansion and Loss of Heterozygosity At Chromosomes 9p and 17p in Premalignant Esophageal (Barrett's) Tissue,” J Natl Cancer Inst 91:2087-95 (1999), which is hereby incorporated by reference in its entirety) or unsorted biopsy material. These samples underwent whole-genome amplification using standard Infinium™ protocols. The resultant product was fragmented to ˜500 bp by enzymatic digestion, precipitated, resuspended in hybridization buffer, denatured, and hybridized to the BeadChip overnight at 48° C. After the overnight incubation, the BeadChip was washed, primer extended, and stained on a Tecan Genesis/EVO robot using a Tecan GenePaint slide processing system. After staining, the BeadChips were washed, immediately coated with a protective reagent, and imaged on Illumina's BeadArray Reader. The image intensities are extracted, and the resultant data analyzed using Illumina's BeadStudio 3.0 software.

Three types of SNP genotyping arrays were employed in the analysis of the above samples: Human-1 BeadChip (109 k loci, n=1 pair); HumanHap300 BeadChip (317 k loci, n=1 pairs); and a non-commercial multi-sample HumanHap300_Pool 0.10 BeadChip (33 k loci, n=20 pairs) which contains a subset (single beadpool) of the overall SNPs from the HumanHap300 BeadChip (33 k) in a multisample format (Peiffer et al., “High-Resolution Genomic Profiling of Chromosomal Aberrations Using Infinium Whole-Genome Genotyping,” Genome Res 16:1136-48 (2006), which is hereby incorporated by reference in its entirety).

Copy number estimates and genotype calls for Illumina BeadChips were calculated using the BeadStudio 2.0 output for raw intensity values and subsequent analyses. Log, ratios for BE samples relative to paired constitutional controls were analyzed per patient for significant regions of copy number loss or gain using the CLAC (Clustering Among Chromosomes) software (Wang et al., “A method For Calling Gains and Losses in Array CGH Data,” Biostatistics 6:45-58 (2005), which is hereby incorporated by reference in its entirety) with a 3 SNP moving window and FDR=0.01. For determination of copy loss or LOH at fragile sites, fragile site names and cytoband locations were downloaded from NCBI MapViewer (Build 36.2). Cytoband positions were obtained from the UCSC Genome Annotation Database. Consensus stem plots were generated using CGH-Explorer (version 2.55) with no smoothing window specified (Lingjaerde et al., “CGH-Explorer: a Program For Analysis of Array-CGH Data,” Bioinformatics 21:821-2 (2005), which is hereby incorporated by reference in its entirety). LOH was determined as a greater than 20% difference in the relative allele frequency between the paired constitutional and BE samples in regions containing at least one informative SNP. Frequency p-values for the all copy number loss, copy number gain, and LOH were evaluated for each chromosome arm using STAC v1.2 with data outputted in binary format per 1 Mb region and 500 permutations per analysis (Diskin et al., “STAC: A Method For Testing the Significance of DNA Copy Number Aberrations Across Multiple Array-CGH Experiments,” Genome Res 16:1149-58 (2006), which is hereby incorporated by reference in its entirety). A p-value cut-off of 0.05 was used for selection of fragile sites with significant regions of loss.

### Example 7

### Quantitative PCR

DNA was extracted from gastric or flow purified BE epithelium, and amplified with quantitative PCR using the Biotage Rotorgene RG-3000 with a protocol adapted from Boehm et al., “Rapid Detection of Subtelomeric Deletion/Duplication by Novel Real-Time Quantitative PCR Using SYBR-Green Dye,” Hum Mutat 23:368-78 (2004), which is hereby incorporated by reference in its entirety. Initial DNA concentrations were measured using the Nanodrop ND-1000 and adjusted to 4 ng/μl. Cycle threshold (Ct) was determined using a Sybr Green fluorescence threshold of 20% and copy numbers were estimated based on amplification relative to a standard curve (2-fold increments ranging from 0.25 to 4 ng) prepared with pooled male genomic DNA (Promega). One Ct difference equates to a loss of one gene copy. Individual runs were normalized using three to four blood samples using 1 ng input genomic DNA to facilitate comparison between reactions with varying efficiencies. Two primers located within region of loss (as determined by SNP genotyping) and one 5′ and one 3′ flanking primer set (on the same chromosome but at least 10 Mb from the fragile site) were used to compare copy number. Primer sequences are provided in Table 5. Copy number estimates were evaluated per chromosome, with flanking regions normalized to 2 copies. The ratio between copy number for BE relative to gastric within the fragile site was then used to identify copy number gains (ratio ≧1.2) or copy number loss (ratio ≦0.8). All samples were run in triplicate.

TABLE 5
Quantitative PCR Primers

| | | | |
|---|---|---|---|
| Fragile | | | |
| site | Location | Forward Primer Sequence | Reverse Primer Sequence |

| | | | | | | |
|---|---|---|---|---|---|---|
| FRA3B | 50.06 | Mb | CAGCCAACGGAGCAG | SEQ ID | TGGGCGATCACCGAAC | SEQ ID |
| | | | | NO: 91 | | NO: 101 |
| FRA3B | 60.45 | Mb | AGCACGGTCTGGGGCT | SEQ ID | TCCTTGCTGCCTGCTCTCT | SEQ ID |
| | | | | NO: 92 | | NO: 102 |
| FRA3B | 60.48 | Mb | TGCCTGAAGTAGGGTTGC | SEQ ID | GGACAGGCCAATGGCA | SEQ ID |
| | | | | NO: 93 | | NO: 103 |
| FRA3B | 60.5 | Mb | TGCCTGCCAAGGGTGT | SEQ ID | CCACCGCCCCAGACTT | SEQ ID |
| | | | | NO: 94 | | NO: 104 |
| FRA3B | 80.51 | Mb | TTAAGGTTCGGGCCATGA | SEQ ID | AGGCATTCGGGCTAGCTG | SEQ ID |
| | | | | NO: 95 | | NO: 105 |
| FRA16D | 65.51 | Mb | AGGCGCTGTTTGAAGGTG | SEQ ID | TTTGCCAAGGCTCTCTCG | SEQ ID |
| | | | | NO: 96 | | NO: 106 |
| FRA16D | 65.78 | Mb | GGGCCCAACAACACTGGA | SEQ ID | GGTCCGGACGAACTGCTG | SEQ ID |
| | | | | NO: 97 | | NO: 107 |
| FRA16D | 77.18 | Mb | AAGCATCTGGGACCACCA | SEQ ID | GGGGATGCAGAGAGTGGA | SEQ ID |
| | | | | NO: 98 | | NO: 108 |
| FRA16D | 77.69 | Mb | GGCTGTCCTCACCGTCA | SEQ ID | GCCAGCCTATGGGTGTGT | SEQ ID |
| | | | | NO: 99 | | NO: 109 |
| FRA16D | 88.17 | Mb | GGGCTGGTGAGCTTGGTG | SEQ ID | AGGCCAGGCAGCAGATGA | SEQ ID |
| | | | | NO: 100 | | NO: 110 |

### Example 8

### Pyrosequencing

LOH at fragile sites was independently confirmed on 20 Barrett's esophagus patients by SNP pyrosequencing. Patient data is summarized in Table 6; DNA from multiple sites at 2 cm intervals within a single BE segment was evaluated when material was available.

TABLE 6
Molecular and histological data for samples analyzed by qPCR and pyrosequencing.

| | | | | | Copy # | LOH | Copy # | LOH |
|---|---|---|---|---|---|---|---|---|
| Patient # | Diagnosis | 9p^{LOH} | 17p^{LOH} | Aneuploid | FRA3B | FRA3B | FRA16D | FRA16D |
| 1 | Metaplasia | ND | ND | No | Gain | 0 of 1 | Normal | 0 of 1 |
| 2 | Metaplasia | No | No | No | Loss | 2 of 4 | Normal | 3 of 4 |
| 3 | Metaplasia | No | No | No | Loss | 1 of 1 | Normal | 0 of 1 |
| 4 | Metaplasia | No | No | No | Loss | 1 of 1 | Loss | 1 of 1 |
| 5 | Metaplasia | No | No | No | Loss | 2 of 4 | Loss | 4 of 4 |
| 6 | Metaplasia | No | No | No | Loss | 0 of 1 | ND | 0 of 1 |
| 7 | Metaplasia | No | No | No | Gain | 1 of 1 | Loss | 1 of 1* |
| 8 | Metaplasia | No | No | No | Loss | 1 of 1 | Loss | 1 of 1 |
| 9 | Metaplasia | Yes | No | No | Loss | 6 of 6 | Normal | 4 of 6 |
| 10 | Metaplasia | Yes | No | No | Loss | 0 of 1 | Normal | 1 of 1 |
| 11 | Metaplasia | Yes | No | No | Loss | 3 of 4 | Loss | 3 of 4 |
| 12 | Indefinite | No | No | No | Normal | 2 of 3 | Normal | 1 of 3 |
| 13 | Indefinite | No | No | No | ND | 2 of 2 | ND | 1 of 2 |
| 14 | Indefinite | No | No | No | Loss | 1 of 3 | Normal | 2 of 3 |
| 15 | Indefinite | No | No | No | Loss | 2 of 2 | ND | 1 of 2 |
| 16 | indefinite | Yes | No | No | ND | 3 of 3 | ND | 3 of 3 |
| 17 | Indefinite | Yes | No | No | Loss | 1 of 1 | Normal | 1 of 1* |
| 18 | Low-Grade | Yes | No | No | Loss | 2 of 2 | ND | 0 of 2 |
| 19 | High-Grade | No | No | No | Loss | 1 of 1 | Loss | 1 of 1 |
| 20 | High-Grade | Yes | No | No | Loss | 3 of 3 | Normal | 2 of 3 |

For LOH, multiple sites were tested from patients, where available, relative to paired gastric constitutional. LOH reflects proportion of sites in which LOH or allelic imbalance was detected relative to the total number of sites tested per patient.
*LOH flank refers to LOH detected in genomic sequence flanking the fragile site, but not within FRA16D itself.
Copy # refers to qPCR data comparing the relative difference in copy number between the BE sample and the paired constitutional samples for 1-2 sites within the respective fragile sites normalized against 2 flanking sites.

For each patient, genomic DNA was extracted from flow sorted endoscopic biopsies and unsorted gastric biopsies, Pyrosequencing (forward, reverse, and sequencing) primers were designed using the PSQ Assay Design 1.0 software to select primers with similar melting temperatures, with products ranging in size from 80 to 120 bp, and overall scores above 85 (See Table 7).

TABLE 7
Pyrosequencing Primers

| Fragile | | | | | |
|---|---|---|---|---|---|
| Site | SNP ID | Forward Primer Sequence | | Reverse Primer Sequence | |
| FRA3B | rs995633 | ACATGGGGGGATAAAAAAGAAGG | SEQ ID | [5′biotin]-AGGATAACAGGAA | SEQ ID |
| | | | NO. 1 | TGGATGGTAAT | NO: 31 |
| FRA3B | rs12635040 | AACCATGACTCAGTCTGAAGTAGC | SEQ ID | [5′biotin]-GGCCACCATATTGGA | SEQ ID |
| | | | NO: 2 | CAGAA | NO: 32 |
| FRA3B | rs213299 | AGGACAGACAAGCAACAAAAATAA | SEQ ID | [5′biotin]-TGTCTTCCCAGCTGA | SEQ ID |
| | | | NO: 3 | ACTTTAGTA | NO: 33 |
| FRA3B | rs2l3418 | [5′biotin]-ATAGCACAGGGACAAC | SEQ ID | TCAAATGAAATCCGTTAGTGTTAA | SEQ ID |
| | | AAAAAATA | NO: 4 | | NO: 34 |
| FRA3B | rs172492 | [5′biotin]-CAAATTTATCCTGGGG | SEQ ID | TGTTCGAAACTGCTCCTGATG | SEQ ID |
| | | CTACG | NO: 5 | | NO: 35 |
| FRA3B | rs213339 | [5′biotin]-TCAGAGATTAGTTCGG | SEQ ID | CCTCCCCATGGTTCTTTGT | SEQ ID |
| | | CTTTGG | NO: 6 | | NO: 36 |
| FRA3B | rs4679531 | CGACTAGAGCTACAAACAAGATTC | SEQ ID | [5′biotin]-TGCACTTTACGTGAC | SEQ ID |
| | | | NO: 7 | AACCTATT | NO: 37 |
| FRA3B | rs12485388 | TCAGGCAAAAAAATGGAAAAGG | SEQ ID | [5′biotin]-TTGGTGGGGGTGTCT | SEQ ID |
| | | | NO: 8 | TTTACTTAT | NO: 38 |
| FRA3B | rs4566489 | GCAGCTACCGAATGATAATAATTG | SEQ ID | [5′biotin]-AACTTTTGTCTGTTC | SEQ ID |
| | | | NO: 9 | GGAATGAG | NO: 39 |
| FRA3B | rs1447981 | [5′biotin]-TGAGCGAAAGTGATQC | SEQ ID | TTTTCTTAGCTCCCATGAAGTCGA | SEQ ID |
| | | AATTACACC | NO: 10 | | NO: 40 |
| FRA3B | rs9863683 | GGAAGCGGTAGGGACTTGAG | SEQ ID | [5′biotin]-GCTCTTCTCCCAAGA | SEQ ID |
| | | | NO: 11 | CATATCCAG | NO: 41 |
| FRA3B | rs939497 | AACATGGGTGCGATAGGTTC | SEQ ID | [5′biotin]-GCAGGCAGTATGTGG | SEQ ID |
| | | | NO. 12 | GTACTTA | NO: 42 |
| FRA3B | rs1716736 | GGAAGGTTGCTTTGGTGATCT | SEQ ID | [5′biotin]-CTGTGCCQACCCCTT | SEQ ID |
| | | | NO: 13 | TACAAAT | NO: 43 |
| FRA3B | rs741891 | GGAGGAGGAGAACATGTGATGTG | SEQ ID | [5′biotin]-TCTGTGTTTGCAGTA | SEQ ID |
| | | | N0: 14 | GGCTTAGGA | NO: 44 |
| FRA3B | rs13098466 | [5′biotin]-GCCTATATTGTGGGTC | SEQ ID | TGATCGTAGCAGAAATTAACAA | SEQ ID |
| | | TTTACTCA | NO: 15 | | NO: 45 |
| FRA16D | rs1079191 | [5′biotin]-ACTGTGTGTTCAGCCG | SEQ ID | AAAGGGCGAGTGAGATAGCAA | SEQ ID |
| | | CTCA | NO: 16 | | NO: 46 |
| FRA16D | rs3419 | [5′biotin]-ACCCAACCACTGTCCC | SEQ ID | GATTTCAAAATGTGGACTCTCAG | SEQ ID |
| | | TGTAAT | NO: 17 | | NO: 47 |
| FRA16D | rs1828518 | CCCTTTGGGTGTTTGTGAA | SEQ ID | [5′biotin]-TCCAGAATCTGTCTC | SEQ ID |
| | | | NO: 18 | CAACAGTT | NO: 48 |
| FRA16D | rs1125671 | TCATAGTTCTCCCCCATCCTG | SEQ ID | [5′biotin]-AACCACCTGGACCCT | SEQ ID |
| | | | NO: 19 | CACTG | NO: 49 |
| FRA16D | rs6564576 | TACCCAGGATGGCTTGATGTT | SEQ ID | [5′biotin]-GAGGCCTGGTTTCAC | SEQ ID |
| | | | NO: 20 | AAATG | NO: 50 |
| FRA16D | rs2042433 | AACCATCCCTGCTCATTCC | SEQ ID | [5′biotin]-TTCTTCTTGGATCCC | SEQ ID |
| | | | NO: 21 | ACATCAC | NO: 51 |
| FRA16D | rs1110559 | GACCACAGCAGGATTTGAATCTAG | SEQ ID | AGAGGATGTAGGGCAGGTAGG | SEQ ID |
| | | | NO: 22 | | NO: 52 |
| FRA16D | rs1125814 | ATTTCAAACATTTGGTGCTAAAAG | SEQ ID | [5′biotin]-TTGCATGCAGTTTAT | SEQ ID |
| | | | NO: 23 | CAAATAGAA | NO: 53 |
| FRA16D | rs982784 | CACGGACCCTTGCCTTTTTAC | SEQ ID | [5′biotin]-GGGAATTTTTATCCT | SEQ ID |
| | | | NO: 24 | GAACTACCG | NO: 54 |
| FRA16D | rs1546360 | [5′biotin]-GCTTTATCAGGTAATT | SEQ ID | TTTGCTGATTCTTGACCTAGAGAG | SEQ ID |
| | | GCAAGTCC | NO: 25 | | NO: 55 |
| FRA16D | rs1966515 | [5′biotin]-CTGCAATCGATCTGAA | SEQ ID | CAACCCCCAAATACACAAACA | SEQ ID |
| | | ATTGACA | NO: 26 | | NO: 56 |
| FRA16D | rs2005036 | GCAACGAGTCCCTTGTTCAG | SEQ ID | [5′biotin]-CCACACTCCTTTTCT | SEQ ID |
| | | | NO: 27 | CCGTTCTTA | NO: 57 |
| FRA16D | rs383362 | [5′biotin]-AGATCCGCAAGAGTAA | SEQ ID | CTGCTTCCCATTGGTACTTAAGAT | SEQ ID |
| | | AGGAAATA | NO: 28 | | NO: 58 |
| FRA16D | rs1876761 | ACGAAGACAAATAGAGGATGAATG | SEQ ID | [5′biotin]-ATGTGCAAAAACAAG | SEQ ID |
| | | | NO: 29 | AAAAGGTAT | NO: 59 |
| FRA16D | rs1472939 | [5′biotin]-GGGGTAACATGACCGG | SEQ ID | GGGCCAAGAGCTCAGGGAT | SEQ ID |
| | | TGACTT | NO: 30 | | NO: 60 |

Pyrosequencing Primers

| | | |
|---|---|---|
| Fragile | | |
| Site | SNP ID | Sequencing Primer Sequence |

| | | | |
|---|---|---|---|
| FRA3B | rs995633 | GCAAGGAATAAAAGGTG | SEQ ID NO: 61 |
| FRA3B | rs12635040 | TCTAATAGAACTTCCTGTGA | SEQ ID NO: 62 |
| FRA3B | rs213299 | GGTCCCTGTCTCCAA | SEQ ID NO: 63 |
| FRA3B | rs2l3418 | AGGATAATTTCACTTTCCA | SEQ ID NO: 64 |
| FRA3B | rs172492 | CGAAACTGCTCCTGA | SEQ ID NO: 65 |
| FRA3B | rs213339 | TTCTTCGCCCATCC | SEQ ID NO: 66 |
| FRA3B | rs4679531 | TATATTTAGTTCAAACCCTG | SEQ ID NO: 67 |
| FRA3B | rs12485388 | ACTATTCTTTTTCAAAGGC | SEQ ID NO: 68 |
| FRA3B | rs4566489 | ATTATCATAAGTGTCACAAT | SEQ ID ND: 69 |
| FRA3B | rs1447981 | TGAAGTCGAAGTTTGTATCT | SEQ ID NO: 70 |
| FRA3B | rs9863683 | CGGTAGGGACTTGAGTTG | SEQ ID NO: 71 |
| FRA3B | rs939497 | TATTAGGGACAGTCACAGA | SEQ ID NO: 72 |
| FRA3B | rs1716736 | TTGCTTTGGTGATCTAGT | SEQ ID NO: 73 |
| FRA3B | rs741891 | CGCATGAAAGACCGC | SEQ ID NO: 74 |
| FRA3B | rs13098466 | CCTACTTCATAGAAGGG | SEQ ID NO: 75 |
| FRA16D | rs1079191 | AATAAAGCCCTGCAGTA | SEQ ID NO: 76 |
| FRA16D | rs3419 | TGTGGACTCTCAGCAG | SEQ ID NO: 77 |
| FRA16D | rs1828518 | TGTTTGTGAAAAGCAAG | SEQ ID NO: 78 |
| FRA16D | rs1125671 | CATCCTGTAAAGGAGCC | SEQ ID NO: 79 |
| FRA16D | rs6564576 | CCTGGGGGTTTTCAA | SEQ ID NO: 80 |
| FRA16D | rs2042433 | CCACCATTTGCTAAATCT | SEQ ID NO: 81 |
| FRA16D | rs1110559 | CAGGTAGGGCTCAGAA | SEQ ID NO: 82 |
| FRA16D | rs1125814 | GTGCTAAAAGACTTAAAGGT | SEQ ID NO: 83 |
| FRA16D | rs982784 | CCCTTGCCTTTTTACC | SEQ ID NO: 84 |
| FRA16D | rs1546360 | CTTGACCTAGAGAGTGCTT | SEQ ID NO: 85 |
| FRA16D | rs1966515 | GGGTCCCCATGAATC | SEQ ID NO: 86 |
| FRA16D | rs2005036 | TCCCTTGTTCAGGCG | SEQ ID NO: 87 |
| FRA16D | rs383362 | AAGATTTTTCACTCTGTTGT | SEQ ID NO: 88 |
| FRA16D | rs1876761 | GTAGAAGATAGGAGGTTGG | SEQ ID NO: 89 |
| FRA16D | rs1472939 | CTAACTCCTGTCTTAGGTAC | SEQ ID NO: 90 |

BLAT search was used to confirm unique sequence for each PCR product. Primers selected to interrogate fragile site FRA3B had a mean HapMap minor allele frequency=0.338 and FRA16D had a mean HapMap minor allele frequency=0.299 (See Table 7). PCR reactions were performed using standard PCR protocol (1×PCR buffer, 1.5 mM MgCl₂, 0.2 ng DNA, 5 mM dNTP, 5 μM primers, 1.25 units AmpliTaq) and amplified using this program: 95° C., 4 min, then 45 cycles at 95° C., 15 see; 57° C. 30 see; 72° C., 15 sec. Pyrosequencing reactions were purified over vacuum prep and mixed with sequencing primer as per manufacturer's instructions. All reactions were performed on the Biotage PSQ HS 96. Allele frequencies were outputted as relative percentages using Biotage PSQ software and genotypes were assigned based on the relative allele frequency between constitutional and experimental samples. Informative calls were evaluated as relative ratios of 50:50+/−10%. Allelic imbalance (AI) was assigned to SNP's which were informative in the constitutional and showed relative allele frequencies of greater than 66:33 to 89:11 in the BE sample. Loss of heterozygosity (LOH) was determined as ratios 90:10 or greater in the BE sample with an informative ratio in the constitutional sample.

### Example 9

### Results

Genome-wide analyses was performed using DNA from 21 patients from the Seattle Barrett's Esophagus Surveillance Program with early molecular-stages of disease (Table 4, group A). These samples included paired gastric and endoscopic BE biopsies, the latter having p16^{LOH} but not p53^{LOH} or aneuploidy (Galipeau et al., “Clonal Expansion and Loss of Heterozygosity At Chromosomes 9p and 17p in Premalignant Esophageal (Barrett's) Tissue,” J Natl Cancer Inst 91:2087-95 (1999), which is hereby incorporated by reference in its entirety). DNA extracted from Ki67 flow sorted epithelium was analyzed for genome-wide abnormalities using either the high density Illumina 33K.

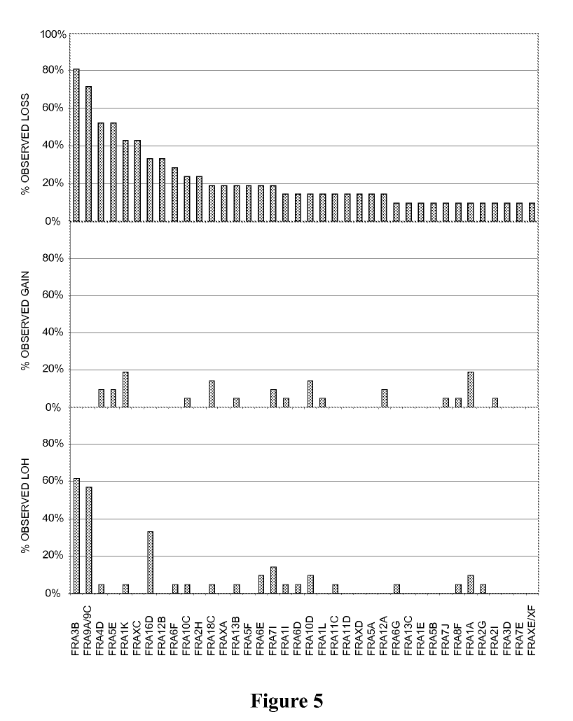

Copy loss, copy gain, and/or LOH were detected in two or more patients at 38 different fragile sites in these samples, as illustrated in FIG. 5. Overall, copy loss was detected at an average of 9.75 fragile sites per patient within this set, with 21 chromosomal fragile sites lost in two or more patients. On average, copy gain was present at an average of 1.3 fragile sites per patient and LOH at 2.6 sites per patient. The most frequent and significant losses were observed at FRA3B (81%; p=0.002), FRA9A/9C (71.4%; p=0.002), FRA4D (52.4%; p=0.002), FRA5F (52.4%; p-value-0.039), FRA1K (42.9%; p-value=0.002), FRAXC (42.9%; p-value=0.004). FRA16D (33.3%; p-value=0.002), and FRA12B (33.3%; p=0.020). Copy gains were observed at low frequency except at FRA19B (42.9%; p=0.01) and FRA8D (33.3%; p=NS). LOH was detected at three fragile sites at high frequency, FRA3B (61.9%; p-value 0.002), FRA9A/9C (57.1%; p-value 0.002), and FRA16D (33.3%; p-value=0.002).

To distinguish between random and significant regions of copy change or LOH within fragile sites, STAC v1.2 was used to identify conserved alterations based on frequency or footprint analysis for all arrays (FIG. 5), as described by Diskin et al., “STAC: A Method for Testing the Significance of DNA Copy Number Abberations Across Multiple Array-CGH Experiments,” Genome Res 16: 1149-58 (2006), which is hereby incorporated by reference in its entirety

Table 8 summarizes the copy number and genotypic alterations detected by analysis of data from this patient set. There were 17 regions of copy loss, gain, or LOH within regions previously identified as fragile sites that were significant by the STAC analysis (Table 8, top group), There were 10 regions of copy loss, gain, or LOH located within 10 Mb of cytoband regions previously identified as fragile sites (Table 8, middle group). These 27 regions in or near fragile sites represented 79.4% of all genomic alterations identified by STAC analysis as significantly altered in these 20 paired patient samples. Significant copy gain events were detected at FRA8D, FRA11H, FRA19B, and FRA22A. Approximately 29.5% of all SNPs detected as having LOH on Illumina 33K arrays were located within just 3 fragile site regions, FRA3 B, FRA9A/9C, and FRA16D.

TABLE 8
Regions of copy change and LOH.

| | | | | | | | | | |
|---|---|---|---|---|---|---|---|---|---|
| | | Start | End | Fragile | | Distance to | | Frequency | Footprint |
| Alteration(s) | Cytoband(s) | (Mb) | (Mb) | Site | Type | Fragile Site | Frequency | p value | p value |

FRAGILE SITES

| | | | | | | | | |
|---|---|---|---|---|---|---|---|---|
| copy loss | 3p14.2 | 59 | 62 | FRA3B | common | 80.00% | 0.001 | 0.004 |
| copy loss | 9p21 | 21 | 23 | FRA9A/9C | rare | 75.00% | 0.001 | 0.008 |
| copy loss | 5p14 | 23 | 30 | FRA5E | common | 55.00% | 0.001 | 0.004 |
| copy loss | 4p15 | 33 | 36 | FRA4D | common | 45.00% | 0.001 | 0.004 |
| copy loss | 5p13 | 29 | 30 | FRA5A | common | 45.00% | 0.0010 | 0.0040 |
| copy loss | 18q22.1 | 63 | 66 | FRA18C | rare | 35.00% | 0.001 | 0.004 |
| copy loss | 16q23.3-24.1 | 76 | 78 | FRA16D | common | 35.00% | 0.001 | 0.004 |
| copy loss | Xq22.1 | 98 | 99 | FRAXC | common | 35.00% | 0.013 | 0.01 |
| copy loss | 1q31 | 185 | 187 | FRA1K | common | 30.00% | 0.001 | 0.006 |
| copy loss | 12q21.3 | 81 | 85 | FRA12B | common | 30.00% | 0.013 | 0.002 |
| copy loss | 7q31.2 | 117 | 118 | FRA7G | common | 20.00% | 0.001 | 0.002 |
| copy loss | 10q21 | 67 | 69 | FRA10C | common | 15.00% | 0.071 | 0.006 |
| copy gain | 8q24.3 | 144 | 146 | FRA8D | common | 25.00% | 0.385 | 0.006 |
| copy gain | 19p13 | 0 | 5 | FRA19B | rare | 35.00% | 0.011 | 0.01 |
| LOH | 9p21 | 21 | 23 | FRA9A/9C | rare | 55.00% | 0.001 | 0.012 |
| LOH | 3p14.2 | 59 | 61 | FRA3B | common | 60.00% | 0.001 | 0.002 |
| LOH | 16q23.3-24.1 | 77 | 79 | FRA16D | common | 35.00% | 0.001 | 0.002 |

NEAR FRAGILE SITES

| | | | | | | | | | |
|---|---|---|---|---|---|---|---|---|---|
| copy loss | 5q14.3 | 83 | 90 | (FRA5B/5D) | common | 1.9 Mb | 50.00% | 0.001 | 0.004 |
| copy loss | 3q25-26.1 | 163 | 170 | (FRA3D) | common | 1.8 Mb | 45.00% | 0.001 | 0.004 |
| copy loss | 13q31.1 | 81 | 86 | (FRA13B) | common | 9.9 Mb | 40.00% | 0.001 | 0.004 |
| copy loss | 11p13 | 38 | 39 | (FRA11E) | common | 1.6 Mb | 35.00% | 0.033 | 0.004 |
| copy loss | 7q21.11 | 84 | 86 | (FRA7E) | common | 4.9 Mb | 35.00% | 0.001 | 0.002 |
| copy loss | 6q21 | 101 | 104 | (FRA6F) | common | 0.8 Mb | 30.00% | 0.045 | 0.006 |
| copy loss | 7p22 | 9 | 11 | (FRA7B) | common | 1.8 Mb | 20.00% | 0.017 | 0.012 |
| copy loss | 11q22.1 | 97 | 98 | (FRA11F) | common | 8.1 Mb | 20.00% | 0.005 | 0.004 |
| copy loss | 11q14.2 | 89 | 90 | (FRA11F) | common | 1.1 Mb | 20.00% | 0.005 | 0.004 |
| copy gain | 10q26.3 | 134 | 135 | (FRA10F) | common | 6 Mb | 15.00% | 0.023 | 0.03 |

NON-FRAGILE SITES

| | | | | | | |
|---|---|---|---|---|---|---|
| copy loss | Xp21.1 | 31 | 33 | 45.00% | 0.001 | 0.006 |
| copy loss | 3p12.1 | 83 | 87 | 40.00% | 0.001 | 0.004 |
| copy loss | Xq25 | 125 | 126 | 40.00% | 0.003 | 0.01 |
| copy loss | 6q22.31 | 124 | 125 | 35.00% | 0.003 | 0.004 |
| copy loss | 21q21.1 | 20 | 22 | 25.00% | 0.01 | 0.04 |
| copy gain | 9q34.2 | 135 | 138 | 20.00% | 0.001 | 0.004 |
| copy gain | 20q13.33 | 60 | 61 | 20.00% | 0.011 | 0.002 |

Shown are significant regions of copy number loss or LOH. Regions within fragile sites (top set), near fragile sites (within 10 Mb, middle set), or at non-fragile sites (bottom set) are shown. Cytogenetic band locations and the chromosome positions (start and end) are noted; the latter indicates the largest extent of the chromosomal region identified by STAC as being statistically altered. The frequency of each alteration is shown. Frequency and footprint p-values were generated in STAC.

To further confirm that these alterations at fragile sites are specific to Barrett's epithelium, two constitutional tissues from eleven patients were analyzed. Copy losses at FRA3B, FRA16D and within significantly altered fragile sites (as listed in Table 8) were observed at similar frequency in BE when compared to either gastric or squamous constitutional samples but many fewer alterations were detected when the reference DNAs were compared against one another. This indicates that the fragile site abnormalities are largely specific to BE and not readily detectable in the normal gastric or squamous epithelium of BE patients.

To confirm the results observed by SNP genotyping, copy number and LOH were evaluated at two fragile sites using q-PCR and pyrosequencing in 20 BE patients previously characterized as having no 17p^{LOH} and no aneuploidy. Patient sample information is provided in Table 6. Fragile sites FRA3B and FRA16D were selected for this study, as the SNP genotyping analysis indicated that these were the sites with the smallest and most consistent consensus regions of copy change (400 kb and 230 kb, respectively); this allowed molecular analysis to be performed with the minimum number of PCR targets for qPCR and pyrosequencing. As shown in Table 6, copy number loss was detected at FRA3B and FRA16D in 83.3% (15 of 18) and 40% (6 of 15) of cases, respectively. Copy number gain was detected at FRA3B in 11.1% (2 of 18) of patients tested. Of note, copy changes were just as frequent in cases without dysplasia as in cases with a maximum diagnosis of indefinite or dysplasia (FRA3B, p>0.29; FRA16D, p>0.44). Pyrosequencing of SNPs spaced within FRA3B or FRA16D, compared to SNPs taken from flanking regions was performed in one to six biopsies per patient, based on availability. When multiple BE samples were analyzed, samples were taken at 2-cm intervals within the BE segment. LOH or allelic imbalance (AI) was detected in at least one SNP and at least one BE biopsy within FRA3 B in 75.0% (15 of 20) of patients (Table 6). At the FRA16D site, we detected LOH or AI in 70.0% of patients (14 of 20). These results confirm the high frequency of copy loss and LOH observed by whole genome analysis.

### Discussion of Examples 5-9

Using SNP genotyping analysis as an effective way to profile for LOH and copy number along with quantitative PCR and pyrosequencing, it has been demonstrated that copy number alteration and/or LOH at chromosomal fragile sites are frequent and early events in BE neoplasia (Table 4, FIG. 5). These changes are present at chromosomal fragile sites that are previously categorized in the literature as both common (e.g., FRA3B, FRA16D, FRA7G) and rare (e.g., FRA9A, FRA18C, and FRA17A) (Schwartz et al., “The Molecular Basis of Common and Rare Fragile Sites,” Cancer Lett 232:13-26 (2006), which is hereby incorporated by reference in its entirety).

Fragile site deletions were observed at much lower frequency in either blood, gastric, or squamous samples from these patients; taken together, these abnormalities are likely not constitutional polymorphisms and are specific to the premalignant columnar BE tissue.

Copy loss and LOH at multiple fragile sites were detectable in early sorted endoscopic BE biopsies by SNP genotyping and further instability was confirmed at FRA3B and FRA16D using PCR and pyrosequencing. While instability at fragile sites has been reported in a number of different cancers, this is the first report showing high frequency of copy loss and LOH within defined regions of multiple chromosomal fragile sites in a premalignant tissue.

It appears that regions of copy loss and LOH in BE can be narrow and well-conserved, in at least a subset of fragile sites, and this is most evident in BE at FRA3B. While published studies of various cancers have reported deletions at FRA3B, these deletions range in size from 300 kb to over 2 Mb (Kameoka et al., “Contig Array CGH at 3 μl 4.2 Points to the FRA3B/FHIT Common Fragile Region as the Target Gene in Diffuse Large B-Cell Lymphoma,” Oncogene 23:9148-54 (2004); Huebner et al., “The Role of the FHIT/FRA3B Locus in Cancer,” Annu Rev Genet 32:7-31 (1998), Sukosd et al., “Deletion of Chromosome 3p14.2-p25 Involving the VHL and FGIT Genes in Conventional Renal Cell Carcinoma,” Cancer Res 63:455-7 (2003) which are hereby incorporated by reference in their entirety) and deletions consistently constrained to the specific sub-region of FRA3B defined herein have not previously been reported in the literature. Both the high frequency and the uniformity of the alterations in BE may reflect a common etiology of genotoxic stress; in BE this is likely oxidative damage, both as a direct effect of bile acids (Jenkins et al., “Deoxycholic Acid (DCA) at Neutral and Acid pH, is Genotoxic to Oesophageal Cells Through the Induction of ROS: the Potential Role of Antioxidants in Barrett's Oesophagus,” Carcinogenesis (2006) and Payne et al., “Deoxycholate Induces Mitochondrial Oxidative Stress and Activates NF-{kappa}B Through Multiple Mechanisms in HCT-116 Colon Epithelial Cells,” Carcinogenesis (2006), which are hereby incorporated by reference in their entirety) and secondarily from oxygen and nitrogen free radicals produced by inflammation. The primary, secondary, and tertiary structure of fragile sites is thought to promote stalling of DNA replication forks, which can induce recombination and strand breakage at fragile sites in these premalignant cells (Glover et al., “Mechanisms of Common Fragile Site Instability,” Hum Mol Genet 14 Spec No 2, R197-205 (2005), which is hereby incorporated by reference in its entirety). Long-term acid suppression, a common therapy for BE, has recently been shown to decrease cellular proliferation via downregulation of Mcm2 expression (Lao-Sirieix et al., “Effect of Acid Suppression on Molecular Predictors For Esophageal Cancer,” Cancer Epidemiol Biomarkers Prev 15:288-93 (2006), which is hereby incorporated by reference in its entirety), and could potentially stabilize fragile sites by alleviating replicative stress. If instability at fragile sites does indeed reflect the history of genotoxic stress in these patients, then it may serve as a biomarker of an individual's history of such damage (Glover et al., “Mechanisms of Common Fragile Site Instability,” Hum Mol Genet 14 Spec No. 2, RI 97-205 (2005), which is hereby incorporated by reference in its entirety). The data herein supports the idea of a specific DNA damage profile, similar to asbestos-related lung cancer. Nymark et al. “Identification of Specific Gene Copy Number Changes in Asbestos-Related Lung Cancer,” Cancer Res 66:5737-43 (2006), which is hereby incorporated by reference in its entirety, recently reported a significant association between copy changes at 11 fragile sites (including FRA19B, FRA22A, and FRA11H) and asbestos-associated alterations by SNP genotyping. Thus, measurement of deletion and LOH at fragile sites merits evaluation as a biomarker of cancer risk in these patients. This biomarker could also play a role as an intermediate indicator of the success of chemopreventative strategies in BE, including the use of non-steroidal anti-inflammatory drugs (NSAIDs) which reduce inflammation and have been shown to be protective for development of esophageal adenocarcinoma (EA) (Vaughan et al., “Non-Steroidal Anti-Inflammatory Drugs and Risk of Neoplastic Progression in Barrett's Oesophagus: a Prospective Study,” Lancer Oncol 6:945-52 (2005), which is hereby incorporated by reference in its entirety).

While we have demonstrated copy loss and LOH at multiple fragile sites, likely due to oxidative injury and inflammation, the potential mechanisms or the functional consequence of instability at these sites have not been elucidated, since little beyond positional information is known about the great majority of the sites. Of those that are well studied, it has been proposed that loss of the tumor suppressor functions of FRA3B and FRA16D could impact neoplastic progression. In fact, copy loss or LOFT at fragile sites, particularly FRA3B, have been previously reported in digestive tract cancers (Kuroki et al., “Common Fragile Genes and Digestive Tract Cancers,” Surg Today 36:1-5 (2006), which is hereby incorporated by reference in its entirety). Although copy loss has also been reported in BE at FRA7G (Miller et al., “(Genomic Amplification of MET With Boundaries Within Fragile Site FRA7G and Upregulation of MET Pathways in Esophageal Adenocarcinoma,” Oncogene 25:409-18 (2006), which is hereby incorporated by reference in its entirety) only copy loss and LOH at FRA7G in the most advanced BE and cancer samples have been observed.

Previous work has shown that inactivation of p16 and p53 are common alterations in BE progression. Indeed, deletion and/or LOH within regions of chromosome arm 9p flanking the p16 locus was observed in the vast majority of samples in this study (FIG. 5), consistent with the previous report that 59% of patients within this BE cohort showed LOH at chromosome arm 9p (Wong et al., “p16(INK4a) Lesions Are Common, Early Abnormalities That Undergo Clonal Expansion in Barrett's Metaplastic Epithelium Evolution of Neoplastic Cell Lineages in Barrett Oesophagus,” Cancer Res 61:8284-9 (2001), which is hereby incorporated by reference in its entirety).

Interestingly, the genomic locus of p16 lies within two fragile sites, FRA9A and FRA9C. It was not determined which of the fragile sites is unstable in these BE samples, since both fragile sites are located at chromosome band 9p21 and were characterized by an in vitro treatment, (i.e. FRA9A from folate deficiency; FRA9C by BrdU). In studies of 18 cell lines, instability at chromosome band 9p21 was reported to occur preferentially at the p16 locus, although instability was also observed in flanking regions. Investigation of the surrounding 10 kb region did not reveal specific breakpoints or regions of di- or tri-nucleotide repeats, which would be susceptible to DNA breakage (Sasaki et al., “Molecular Processes of Chromosome 9p21 Deletions in Human Cancers,” Oncogene 22:3792-8 (2003), which is hereby incorporated by reference in its entirety). Likewise, previous data suggests that loss at FRA17A may be a surrogate measure of instability at the proximal p53, already a well-known player in BE disease progression. However, FRA17A is considered to be a rare fragile site (present in less than 5% of the population) and no functional connection between loss at FRA17A and p53 have been reported, so it is unclear whether instability at FRA17A and p53 are associated. The loss at FRA10C was intriguing, since the gene affected is most likely due to loss of CTNNA3, the catenin (cadherin associated protein) alpha 3, a member of the Wnt signaling pathway, also recently reported to be involved in BE neoplastic progression (Clement et al., “Alterations of the Wnt Signaling Pathway During the Neoplastic Progression of Barrett's Esophagus,” Oncogene 25:3084-92 (2006), which is hereby incorporated by reference in its entirety).

FRA3B is of particular interest, as it is one of the most frequently lost sites in cancers, with loss of FHIT protein expression observed in about 60% of human tumors, in up to 50% of esophageal and stomach carcinomas (Ohta et al., “The FHIT Gene, Spanning the Chromosome 3p14.2 Fragile Site and Renal Carcinoma-Associated t(3;8) Breakpoint, is Abnormal in Digestive Tract Cancers,” Cell 84:587-97 (1996), which is hereby incorporated by reference in its entirety) and in approximately 31% of precancerous lesions including Barrett's metaplasia, bronchial lesions, breast ductal CIS, cervical lesions, colorectal adenomas, and aberrant colorectal crypt foci (Ishii et al., “Potential Cancer Therapy With the Fragile Histidine Triad Gene Review of the Preclinical Studies,” Jama 286:2441-9 (2001), which is hereby incorporated by reference in its entirety). Thus far, reports have linked copy change or LOH at two specific fragile sites in EA, FRA7G and FRA3B (Miller et ah, “Genomic Amplification of MET With Boundaries Within Fragile Site FRA7G and Upregulation of MET Pathways in Esophageal Adenocarcinoma,” Oncogene 25:409-18 (2006); Zou et al., “FHIT Gene Alterations in Esophageal Cancer and Ulcerative Colitis (UC),” Oncogene 15:101-5 (1997); Menin et al., “Anomalous Transcripts and Allelic Deletions of the FHIT Gene in Human Esophageal Cancer,” Cancer Genet Cytogenet 119:56-61 (2000); Mori et al., “Altered Expression of Fhit in Carcinoma and Precarcinomatous Lesions of the Esophagus,” Cancer Res 60:1177-82 (2000), which are hereby incorporated by reference in their entirety). Although Zou et. al (1997) reported only 20% LOH at FRA3B in EA, using Southern hybridization Michael et al., “Frequent Deletions of FHIT and FRA3B in Barrett's Metaplasia and Esophageal Adenocarcinomas,” Oncogene 15:1653-9 (1997), which is hereby incorporated by reference in its entirety, found deletions in 60% of EA and 30% of BE metaplasias. Similarly, Menin et al., “Anomalous Transcripts and Allelic Deletions of the FHIT Gene in Human Esophageal Cancer,” Cancer Genet Cytogenet 119:56-61 (2000), which is hereby incorporated by reference in its entirety, observed high frequency of alterations at FHIT, with aberrant transcripts or LOH detected in 77% (30/39) of EA samples tested. Re-introduction of FHIT in heterozygous knock-out mice demonstrated a dramatic decrease in carcinogen (NMBA) induced esophageal tumors (Dumon et al., “FHIT Gene Therapy Prevents Tumor Development in Fhit-Deficient Mice,” Proc Natl Acad Sci USA 98:3346-51 (2001), which is hereby incorporated by reference in its entirety). Similarly, ectopic expression of WWOX (located at FRA16D) suppressed tumorigenicity of lung cancer cells in nude mice and inhibited growth of cancer cell lines in vitro (Fabbri et al., “WWOX Gene Restoration Prevents Lung Cancer Growth in vitro and in vivo,” Proc Natl Acad Sci USA 102:15611-6 (2005), which is hereby incorporated by reference in its entirety). Investigation of the impact of instability at FRA3B, FRA16D, and other fragile sites on cellular pathways to regulate cancer growth and/or progression will be enhanced by the continued molecular characterization of these dynamic chromosomal regions.

The data herein suggests that the genetic mechanisms regulating responses to replicative stress are impaired or overwhelmed in this inflamed environment prior to development of aneuploidy (or dysplasia), and instability at or near fragile sites constitutes ˜80% of the statistically significant and conserved copy number loss events within early BE samples (Table 8). Using the STAC software, highly unstable 1 to 7 Mb regions have been defined within the larger cytobands (Table 4) which will further facilitate identification of genes within fragile sites and the sequence analysis of potential breakpoints or unstable nucleotide repeats.

The studies described herein have demonstrated that deletion at FRA3B and multiple other fragile sites can be detected in Barrett's epithelium using flow sorted endoscopic biopsies by SNP genotyping arrays, qPCR, and pyrosequencing, suggesting that these lesions are present at high frequency and are detectable using diverse methods. In addition, increasing frequency of copy loss and LOH with disease progression supports clonal expansion within the BE segment (Maley et al. “The Combination of Genetic Instability and Clonal Expansion Predicts Progression to Esophageal Adenocarcinoma,” Cancer Res 64:7629-33 (2004), which is hereby incorporated by reference in its entirety). Deletion at fragile sites in BE can serve as an informative biomarker of the extent of genomic damage and therefore cancer risk, in combination with other previously reported molecular markers.

### Example 10

### Diagnostic Clinical Assay for Identification of FRA3B/FHIT Deletions

Whole-genome SNP arrays for array-CGH (comparative genomic hybridization) have been used to investigate chromosomal and genotypic alterations in flow sorted epithelium from endoscopic and surgical BE samples, relative to paired blood or gastric constitutional. As described supra, whole genome analysis was performed on 26 patients using commercially available high density microarray platforms from Affymetrix, Inc. and Illumina, Inc. As expected, loss at p16, a cell cycle inhibitor previously shown to be frequently lost in Barrett's esophagus, was observed in the majority of samples (19 of 26 patients; 73%). However, it was surprising to observe a loss in a number of fragile sites (see FIG. 5), including FRA3B, FRA16D, FRA13B, and a number of additional fragile sites at lower frequency, most of which have not yet been shown to be involved in neoplastic progression of BE. Deletion at fragile sites 3B, 13B, or 1K was observed in 100% (26 of 26) of patients tested. These alterations were detected only in Barrett's epithelium and not gastric or normal squamous epithelium of the same patients, verifying that these changes are specific to the metaplastic columnar tissue. The instability at multiple fragile site suggests that these changes result from specific DNA damage and replication stress sustained as a result of bile and acid reflux.

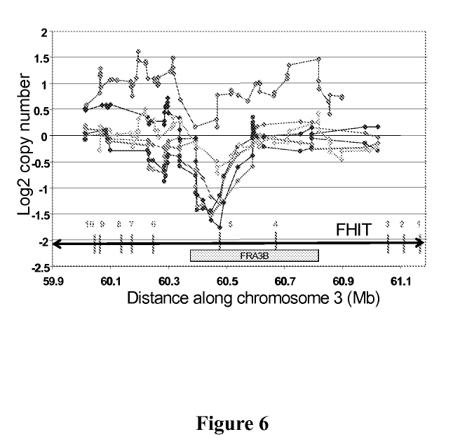

FIG. 6 illustrates that high resolution analysis of the most frequently altered fragile site, FRA3B, defines a surprisingly small loci of involvement. Deletions consistently constrained to these specific subregions of the respective fragile sites have not previously been reported in the literature.

Using the Affymetrix and Illumina genotyping arrays, it was observed that the deletion in the FRA3B site is constrained to approximately a 250 kb deletion on chromosome band 3p14 which corresponds to a region within the FHIT gene, including portions of intron 4, exon 5, and at least a portion of intron 5 of the FHIT gene (FIG. 6). These copy losses can be confirmed using an independent technique, quantitative PCR (q-PCR), which further demonstrates that deletion at FRA3B is a practical biomarker of cancer risk in BE patients. Loss at this site by qPCR analysis has also been observed in two BE cell lines previously characterized, QhTRT and ChTRT (Palanca-Wessels et al., “Extended Lifespan of Barrett's Esophagus Epithelium Transduced with the Human Telomerase Catalytic Subunit: a Useful In Vitro Model,” Carcinogenesis, 24:1183-1190 (2003), which is hereby incorporated by reference in its entirety) when compared against copy number for flanking sequence on chromosome 3.

Thus, the studies described herein have demonstrated the presence of a small, uniform deletion in 80.7% of BE samples (n=26 patients), which makes this an unusually consistent genetic marker of a pre-malignant tissue. This represents an opportunity for detection of the presence of BE in patients by a molecular diagnostic test without the use of conventional endoscopic examination and biopsy material. While q-PCR and whole genome arrays are useful for detecting fragile site loss in endoscopic biopsies in BE, the ability of these assays to detect copy loss in mixed cell populations is limited since copy number alterations would only be detected above background if they were present within a significant proportion of the sample, (i.e. no less than 20-25%). The detection of a small deletion in FHIT within a mixed population of cells, or better yet, as a rare event in a background of large numbers of normal DNA sequence, will serve as a sensitive diagnostic marker for the presence of BE. This test can be applied to esophageal brushings, and potentially to the detection of small numbers of molecules of DNA copies from BE cells that are present in circulating blood.

Currently, the field of “rare event” detection in the cancer prevention field has focused on three modes of detection: 1) rare circulating tumor cells or “CTCs” from cancer; 2) circulating RNA, and 3) circulating DNA. There have been multiple reports of circulating cancer cells in blood, and their isolation by magnetic beads and/or cell sorting, usually based on detecting expression of cytokeratin, a cell component not ordinarily present in blood cells (Cristofanilli et al., “Circulating Tumor Cells: a Novel Prognostic Factor for Newly Diagnosed Metastatic Breast Cancer,” J Clin Oncol. 23:1420-1430 (2005), which is hereby incorporated by reference in its entirety). Whole cancer cells are present in blood, however, only when a cancer is advanced and/or metastatic. Thus, it is unlikely to be of use in the context of a preneoplastic disease. RT-PCR has been used to detect cancer-specific RNA transcripts within peripheral blood. However, this assay is dependent upon timely processing since RNA is less stable than DNA (Benoy et al., “Detection of Circulating, Tumour Cells in Blood by Quantitative Real-Time RT-PCR: Effect of Pre-analytical Time,” Clin Chem Lab Med. 44:1082-1087 (2006), and Peck et al., “Detection and Quantitation of Circulating Cancer Cells in the Peripheral Blood of Lung Cancer Patients,” Cancer Res. 58:2761-2765 (1998), which are hereby incorporated by reference in their entirety). Finally, the most successful strategies have been based on detecting in circulation, DNA that contains a mutated sequence that originated in cancer cells. This concept is based on the fact that a novel sequence, e.g., a mutation in cancer that is not present in constitutional DNA, can be detected even at very low frequencies using PCR. This is presently most successfully used to monitor minimal residual disease in patients with hematologic malignancies (reviewed by Schuler and Dolken, “Detection and Monitoring of Minimal Residual Disease by Quantitative Real-Time PCR,” Clin Chim Aca. 363:147-156 (2006), which is hereby incorporated by reference in its entirety), although it has also been used to detect mutant DNA from solid cancers within the circulation (Ashida et al., “Detection of Circulating Cancer Cells with von Hippel-lindau Gene Mutation in Peripheral Blood of Patients with Renal Cell Carcinoma,” Clin Cancer Res. 6:3817-3822 (2000); Lindforss et al., “Persistence of K-ras Mutations in Plasma After Colorectal Tumor Resection. Anticancer Res. 25:657-661 (2005); and Burchill and Selby. “Molecular Detection of Low-Level Disease in Patients with Cancer,” J Pathol, 190:6-14 (2000), which are hereby incorporated by reference in their entirety). This method does, however, require knowing in advance what novel sequence is characteristic of the neoplastic cells that is not present in normal cells.

There are several challenges to the development of robust assays that are sufficiently sensitive to detect rare sequences in a background of normal sequence: technical challenges in detecting rare sequences against a normal background and the challenge of defining a novel sequence in the target cells of interest. While some genetic alterations have been reported to be present in premalignant BE tissue (Galipeau et al., “Clonal Expansion and Loss of Heterozygosity at Chromosomes 9p and 17p in Premalignant Esophageal (Barrett's) Tissue,” J Natl Cancer Inst. 91:2087-2095 (1999); Gonzalez et al., “Mutation Analysis of the p53, APC, and p16 Genes in the Barrett's Esophagus, Dysplasia, and Adenocarcinoma,” J Clin Pathol. 50:212-217 (1997); and Koppert et al., “The Molecular Biology of Esophageal Adenocarcinoma,” J Surg Oncol. 92:169-190 (2005), which are hereby incorporated by reference in their entirety), none have thus far been present at a sufficiently high frequency to enable their use in screening of at-risk populations. Herein is described a genetic lesion that is present in ˜80% of BE and this is thus a unique case in which it is known in advance, with ˜80% sensitivity, the identity of a DNA sequence abnormality in the target cells. With the exception of viral sequences (such as HPV in cervical cells), such a genetic marker of preneoplastic cells has heretofore not been available for a premalignant tissue.

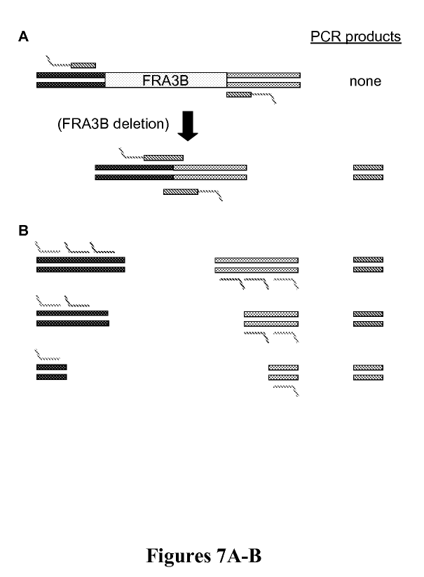

The regions of deletion at FRA3B that have been identified in BE are remarkably small and well conserved across patients. This is likely due to the common nature of the genotoxic environment (acid, bile and inflammation) in reflux disease in these patients and its effect on DNA replication of a specific fragile sequence. The reproducibility of the deletion in FRA3B seen in BE makes it possible to design a PCR strategy for its detection (FIG. 7). In this scheme, PCR products can only be produced if the deletion is present, as otherwise the primer pairs are too far apart to allow PCR product to be formed.

The deletion in FRA3B has been identified by amplifying across the surrounding genomic region (150 to 200 kb when not deleted) using long-extension PCR. The normal DNA sequence cannot be amplified by the PCR reaction, as the primers are too far apart to allow synthesis of a PCR product (FIG. 7). In the presence of a deletion within the region of the FRA3B site, as has been observed in BE, the primers can be brought close enough to enable extension. If the method of long extension PCR is used, the primers can be as far as 30 kb apart and product can still be formed. The sequence containing the deletion can be detected as a very rare event in a mixed population, as only the deletion sequence is a substrate for the PCR reaction.

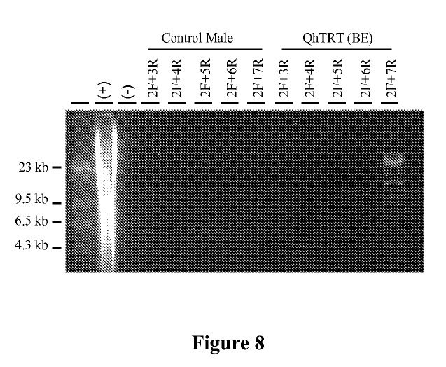

A PCR based assay to amplify a consensus region of the fragile site deletions detected in the previous studies was optimized. Since PCR works by the selective hybridization of primers and extension across a stretch of DNA of limited size this assay is sensitive to populations present in a subset of a sample—in principal a single copy present within peripheral blood or a small subset of cells within a cytologic brush sample. A product from long extension PCR will be produced only if a deletion is present that brings the primer sites to within 30 kb of each other (without the deletion, the primers are approximately 250 kb apart, a distance too large to extend across even with specially adapted “long extension” polymerases). Because of variability in the size of the deleted region (FIG. 6), 4-8 staggered primer pairs (at 25 kb intervals) may be necessary to insure that one of the primer pairs is brought to within 30 kb of each other. An example is shown in FIG. 8, in which one of 5 primer pairs amplified a region of the FRA3B site containing the deletion in BE cells.

This assay can be applied for high-throughput analysis, has a minimal DNA requirement, and most importantly, would allow for detection of deletions present within mixed cell populations or rare copies of DNA admixed with normal sequence. The assay can thus be applied to specimens obtained in minimally invasive procedures, such as cytological brushes or ultrathin fiber optic endoscopic instruments rather than the standard endoscopic protocol that requires biopsy using large diameter endoscopes, and DNA samples will be satisfactory even if only very few BE cells are recovered. The PCR reaction can be optimized to use starting amounts of less than 1 ng of genomic DNA per PCR reaction (roughly 150 cell equivalents). Varying proportions of normal (leukocyte) DNA will be added back to ensure the assay is not affected by the presence of normal cells within the sample.

Genomic DNA will be extracted from the blood and cytologic brush samples using the DNeasy Blood & Tissue kit (Qiagen #69504), which can be used for samples as small as 100 cells. Purified DNA will be quantitated using a Nanodrop ND-1000 device Nanodrop Technologies) to check DNA quality and estimate concentration. Mixtures of genomic DNA isolated from pre characterized BE cell lines (QhTRT and ChTRT), which contain the FRA3B deletion (Palanca-Wessels et al., “Extended Lifespan of Barrett's Esophagus Epithelium Transduced with the Human Telomerase Catalytic Subunit: a Useful In Vitro Model,” Carcinogenesis, 24:1183-1190 (2003), which is hereby incorporated by reference in its entirety) with a control normal DNA (Promega genomic male DNA #G1471) to evaluate the threshold for sensitivity of this assay. PCR reactions will be set up in 25 μl volumes using 1 ng or less of total input DNA in a standard long extension protocol with the Expand Long Template PCR system (Roche #11-681-842-001) as per manufacturer's instructions and visualized by gel electrophoresis.

For control experiments, DNA mixtures will be set up using ratios of cell equivalents from BE cell lines and blood in increments ranging from 1:10 to 1:10,000,000. Experiments will also be performed on samples isolated from patients with chronic gastro-esophageal reflux disease (GERD) to confirm discrimination between patients with GERD vs. BE. Finally, the prospective BE biopsies, esophageal brushings and blood samples will be tested to verify that deletions identified within BE preneoplastic tissue can also be detected in mixed cell populations from esophageal brushings or blood samples from the same individual.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of diagnosing a premalignant condition in a human subject, said method comprising:
providing a genomic DNA sample from a human subject;
determining the presence of chromosomal alterations at one or more fragile chromosome sites selected from the group consisting of FRA13B, FRA16D, FRA1K, FRA11D, FRA12B, FRA20A, FRA10C, FRA10D, FRA7I, FRA9A/9C, FRA4D, FRA5E, FRAXC, and FRA18C in the genomic DNA sample; and
diagnosing, based on said determining, the premalignant condition in the human subject.

2. The method according to claim 1, wherein the premalignant condition is associated with chronic inflammation.

3. The method according to claim 1, wherein the premalignant condition is associated with reactive oxygen species.

4. The method according to claim 1, wherein the premalignant condition is associated with a tissue selected from the group consisting of prostate, breast, lung, esophagus, and bladder tissue.

5. The method according to claim 4, wherein the premalignant condition is Barrett's Esophagus.

6. The method according to claim 1, wherein said chromosomal alteration is selected from the group consisting of genomic copy number gain, genomic copy number loss, and loss of heterozygosity.

7. The method according to claim 1 further comprising:
determining the presence of a genomic deletion in a FRA3B fragile chromosome site in the genomic DNA sample.

8. The method according to claim 7, wherein the genomic deletion in the FRA3B fragile chromosome site comprises a 250 kB region of the FHIT gene including at least a portion of intron 4 and at least a portion of intron 5 of the FHIT gene.

9. The method according to claim 1, wherein the sample comprises genomic DNA isolated from cells of the premalignant condition.

10. The method according to claim 9, wherein the cells of the premalignant condition are collected using a cytological brush.

11. The method according to claim 1, wherein the sample comprises genomic DNA isolated from a peripheral blood sample.

12. The method according to claim 1, wherein said determining further comprises:
comparing the presence of chromosomal alterations at one or more fragile sites in the genomic sample to the presence of chromosomal alterations at corresponding fragile sites in a genomic sample not associated with the premalignant condition.

13. The method according to claim 1, wherein said determining comprises:
detecting chromosomal alterations using one or more assays selected from the group consisting of a nucleic acid amplification assay, a hybridization assay, and a nucleic acid sequencing assay.

14. The method according to claim 13, wherein said detecting is carried out with a nucleic acid amplification assay in the form of a long-extension polymerase chain reaction.

15. The method according to claim 13, wherein the one or more assays are adapted for high-throughput analysis.

16. The method according to claim 1 further comprising:
establishing a prognosis for the premalignant condition in the human subject based on said determining and diagnosing.

17. The method according to claim 1 further comprising:
establishing a therapeutic regimen for the premalignant condition in the human subject based on said determining and diagnosing.

18. The method according to claim 17 further comprising:
repeating said determining and said diagnosing during progression of the premalignant condition and adjusting the therapeutic regimen based on said repeating said determining and said diagnosing.

19. The method according to claim 17 further comprising:
repeating said determining and said diagnosing after establishing a therapeutic regimen and ascertaining the human subject's response to said therapeutic regimen.

20. A diagnostic kit comprising
a detection assay for determining, in a DNA sample which is from a human subject, chromosomal alterations at one or more sites within one or more fragile chromosome sites selected from the group consisting of FRA13B, FRA16D, FRA1K, FRA11D, FRA12B, FRA20A, FRA10C, FRA10D, FRA7I, FRA9A/9C, FRA4D, FRA5E, FRAXC, and FRA18C.

21. The diagnostic kit according to claim 20, wherein said detection assay comprises one or more oligonucleotide primer sets wherein each primer set is designed to detect a chromosomal alteration in one or more specific fragile chromosome sites.

22. The diagnostic kit according to claim 20 further comprising:
a detection assay for detecting a genomic deletion in the FRA3B fragile site.

23. A method of diagnosing a premalignant condition in a human subject, said method comprising:
providing a genomic DNA sample from a human subject;
determining the presence of a genomic deletion at the FRA3B fragile site, wherein said deletion comprises a 250 kB region of the FHIT gene including at least a portion of intron 4 and at least a portion of intron 5 of the FHIT gene; and
diagnosing, based on said determining, the premalignant condition in the human subject.

24. The method according to claim 23, wherein the premalignant condition is associated with chronic inflammation.

25. The method according to claim 23, wherein the premalignant condition is associated with reactive oxygen species.

26. The method according to claim 23, wherein the premalignant condition is associated with a tissue selected from the group consisting of prostate, breast, lung, esophagus, and bladder tissue.

27. The method according to claim 26, wherein the premalignant condition is Barrett's Esophagus.

28. The method according to claim 23, wherein the sample comprises genomic DNA isolated from cells of the premalignant condition.

29. The method according to claim 28, wherein the cells of the premalignant condition are collected using a cytological brush.

30. The method according to claim 23, wherein the sample comprises genomic DNA isolated from a peripheral blood sample.

31. The method according to claim 23 wherein said determining further comprises:
comparing the presence of a genomic deletion at the FRA3B fragile sites in the genomic sample to the presence of a genomic deletion at the FRA3B fragile site in a genomic sample not associated with the premalignant condition.

32. The method according to claim 23, wherein said determining comprises:
detecting a genomic deletion at the FRA3B fragile site using one or more assays selected from the group consisting of a nucleic acid amplification assay, a hybridization assay, and a nucleic acid sequencing assay.

33. The method according to claim 32, wherein said determining is carried out with a nucleic acid amplification assay in the form of a long-extension polymerase chain reaction comprising oligonucleotide primers having sequence complementarity to at least a portion of intron 4 and at least a portion of intron 5 of the FHIT gene.

34. The method according to claim 33, wherein the presence of an amplification product resulting from the long-extension polymerase chain reaction indicates a genomic deletion at the FRA3B fragile site.

35. The method according to claim 32, wherein the one or more assays are adapted for high-throughput analysis.

36. The method according to claim 23 further comprising:
establishing a prognosis for the premalignant condition in the human subject based on said determining and diagnosing.

37. The method according to claim 23 further comprising
establishing a therapeutic regimen for the premalignant condition in the human subject based on said determining and diagnosing.

38. The method according to claim 37 further comprising:
repeating said determining and said diagnosing during progression of the premalignant condition and
adjusting the therapeutic regiment based on said repeating said determining and said diagnosing.

39. The method according to claim 37 further comprising:
repeating said determining and said diagnosing after establishing a therapeutic regimen and
ascertaining the human subject's response to said therapeutic regimen.

40. A diagnostic kit comprising:
a detection assay for determining in a DNA sample, which is from a human subject, a genomic deletion in the FRA3B fragile site, wherein the genomic deletion comprises a 250 kb region of the FHIT gene including at least a portion of intron 4 and at least a portion of intron 5 of the FHIT gene.

41. The diagnostic kit according to claim 40, wherein said detection assay comprises one or more oligonucleotide primer sets wherein the first primer of the primer set has sequence complementarity to at least a portion of intron 4 of the FHIT gene and the second primer of the primer set has sequence complementarity to at least a portion of intron 5 of the FHIT gene.


SEQUENCE LISTING (116 sequences)

SEQ ID NO: 1 - DNA, 22 bp, Artificial
  primer
acatggggggataaaaaagaag

SEQ ID NO: 2 - DNA, 24 bp, Artificial
  primer
aaccatgactcagtctgaagtagc

SEQ ID NO: 3 - DNA, 24 bp, Artificial
  primer
aggacagacaagcaacaaaaataa

SEQ ID NO: 4 - DNA, 24 bp, Artificial
  primer
atagcacagggacaacaaaaaata

SEQ ID NO: 5 - DNA, 21 bp, Artificial
  primer
caaatttatcctggggctacg

SEQ ID NO: 6 - DNA, 22 bp, Artificial
  primer
tcagagattagttcggctttgg

SEQ ID NO: 7 - DNA, 24 bp, Artificial
  primer
cgactagagctacaaacaagattc

SEQ ID NO: 8 - DNA, 22 bp, Artificial
  primer
tcaggcaaaaaaatggaaaagg

SEQ ID NO: 9 - DNA, 24 bp, Artificial
  primer
gcagctaccgaatgataataattg

SEQ ID NO: 10 - DNA, 24 bp, Artificial
  primer
tgagcgaaagtgatcaattacacc

SEQ ID NO: 11 - DNA, 20 bp, Artificial
  primer
ggaagcggtagggacttgag

SEQ ID NO: 12 - DNA, 20 bp, Artificial
  primer
aacatgggtgcgataggttc

SEQ ID NO: 13 - DNA, 21 bp, Artificial
  primer
ggaaggttgctttggtgatct

SEQ ID NO: 14 - DNA, 23 bp, Artificial
  primer
ggaggaggagaacatgtgatgtg

SEQ ID NO: 15 - DNA, 24 bp, Artificial
  primer
gcctatattgtgggtctttactca

SEQ ID NO: 16 - DNA, 20 bp, Artificial
  primer
actgtgtgttcagccgctca

SEQ ID NO: 17 - DNA, 22 bp, Artificial
  primer
acccaaccactgtccctgtaat

SEQ ID NO: 18 - DNA, 19 bp, Artificial
  primer
ccctttgggtgtttgtgaa

SEQ ID NO: 19 - DNA, 21 bp, Artificial
  primer
tcatagttctcccccatcctg

SEQ ID NO: 20 - DNA, 21 bp, Artificial
  primer
tacccaggatggcttgatgtt

SEQ ID NO: 21 - DNA, 19 bp, Artificial
  primer
aaccatccctgctcattcc

SEQ ID NO: 22 - DNA, 24 bp, Artificial
  primer
gaccacagcaggatttgaatctag

SEQ ID NO: 23 - DNA, 24 bp, Artificial
  primer
atttcaaacatttggtgctaaaag

SEQ ID NO: 24 - DNA, 21 bp, Artificial
  primer
cacggacccttgcctttttac

SEQ ID NO: 25 - DNA, 24 bp, Artificial
  primer
gctttatcaggtaattgcaagtcc

SEQ ID NO: 26 - DNA, 23 bp, Artificial
  primer
ctgcaatcgatctgaaattgaca

SEQ ID NO: 27 - DNA, 20 bp, Artificial
  primer
gcaacgagtcccttgttcag

SEQ ID NO: 28 - DNA, 24 bp, Artificial
  primer
agatccgcaagagtaaaggaaata

SEQ ID NO: 29 - DNA, 24 bp, Artificial
  primer
acgaagacaaatagaggatgaatg

SEQ ID NO: 30 - DNA, 22 bp, Artificial
  primer
ggggtaacatgaccggtgactt

SEQ ID NO: 31 - DNA, 24 bp, Artificial
  primer
aggataacaggaatggatggtaat

SEQ ID NO: 32 - DNA, 20 bp, Artificial
  primer
ggccaccatattggacagaa

SEQ ID NO: 33 - DNA, 24 bp, Artificial
  primer
tgtcttcccagctgaactttagta

SEQ ID NO: 34 - DNA, 24 bp, Artificial
  primer
tcaaatgaaatccgttagtgttaa

SEQ ID NO: 35 - DNA, 21 bp, Artificial
  primer
tgttcgaaactgctcctgatg

SEQ ID NO: 36 - DNA, 19 bp, Artificial
  primer
cctccccatggttctttgt

SEQ ID NO: 37 - DNA, 23 bp, Artificial
  primer
tgcactttacgtgacaacctatt

SEQ ID NO: 38 - DNA, 24 bp, Artificial
  primer
ttggtgggggtgtcttttacttat

SEQ ID NO: 39 - DNA, 23 bp, Artificial
  primer
aacttttgtctgttcggaatgag

SEQ ID NO: 40 - DNA, 24 bp, Artificial
  primer
ttttcttagctcccatgaagtcga

SEQ ID NO: 41 - DNA, 24 bp, Artificial
  primer
gctcttctcccaagacatatccag

SEQ ID NO: 42 - DNA, 22 bp, Artificial
  primer
gcaggcagtatgtgggtactta

SEQ ID NO: 43 - DNA, 21 bp, Artificial
  primer
ctgtgccacccctttacaaat

SEQ ID NO: 44 - DNA, 24 bp, Artificial
  primer
tctgtgtttgcagtaggcttagga

SEQ ID NO: 45 - DNA, 24 bp, Artificial
  primer
tgatcgtagcagacaaattaacaa

SEQ ID NO: 46 - DNA, 21 bp, Artificial
  primer
aaagggcgagtgagatagcaa

SEQ ID NO: 47 - DNA, 24 bp, Artificial
  primer
gatttccaaaatgtggactctcag

SEQ ID NO: 48 - DNA, 23 bp, Artificial
  primer
tccagaatctgtctccaacagtt

SEQ ID NO: 49 - DNA, 20 bp, Artificial
  primer
aaccacctggaccctcactg

SEQ ID NO: 50 - DNA, 20 bp, Artificial
  primer
gaggcctggtttcacaaatg

SEQ ID NO: 51 - DNA, 22 bp, Artificial
  primer
ttcttcttggatcccacatcac

SEQ ID NO: 52 - DNA, 21 bp, Artificial
  primer
agaggatgtagggcaggtagg

SEQ ID NO: 53 - DNA, 24 bp, Artificial
  primer
ttgcatgcagtttatcaaatagaa

SEQ ID NO: 54 - DNA, 24 bp, Artificial
  primer
gggaatttttatcctgaactaccg

SEQ ID NO: 55 - DNA, 24 bp, Artificial
  primer
tttgctgattcttgacctagagag

SEQ ID NO: 56 - DNA, 21 bp, Artificial
  primer
caacccccaaatacacaaaca

SEQ ID NO: 57 - DNA, 24 bp, Artificial
  primer
ccacactccttttctccgttctta

SEQ ID NO: 58 - DNA, 24 bp, Artificial
  primer
ctgcttcccattggtacttaagat

SEQ ID NO: 59 - DNA, 24 bp, Artificial
  primer
atgtgcaaaaacaagaaaaggtat

SEQ ID NO: 60 - DNA, 19 bp, Artificial
  primer
gggccaagagctcagggat

SEQ ID NO: 61 - DNA, 17 bp, Artificial
  primer
gcaaggaataaaaggtg

SEQ ID NO: 62 - DNA, 20 bp, Artificial
  primer
tctaatagaacttcctgtga

SEQ ID NO: 63 - DNA, 15 bp, Artificial
  primer
ggtccctgtctccaa

SEQ ID NO: 64 - DNA, 19 bp, Artificial
  primer
aggataatttcactttcca

SEQ ID NO: 65 - DNA, 15 bp, Artificial
  primer
cgaaactgctcctga

SEQ ID NO: 66 - DNA, 15 bp, Artificial
  primer
ttcttcgccccatcc

SEQ ID NO: 67 - DNA, 20 bp, Artificial
  primer
tatatttagttcaaaccctg

SEQ ID NO: 68 - DNA, 19 bp, Artificial
  primer
actattctttttcaaaggc

SEQ ID NO: 69 - DNA, 20 bp, Artificial
  primer
attatcataagtgtcacaat

SEQ ID NO: 70 - DNA, 20 bp, Artificial
  primer
tgaagtcgaagtttgtatct

SEQ ID NO: 71 - DNA, 18 bp, Artificial
  primer
cggtagggacttgagttg

SEQ ID NO: 72 - DNA, 19 bp, Artificial
  primer
tattagggacagtcacaga

SEQ ID NO: 73 - DNA, 18 bp, Artificial
  primer
ttgctttggtgatctagt

SEQ ID NO: 74 - DNA, 15 bp, Artificial
  primer
cgcatgaaagaccgc

SEQ ID NO: 75 - DNA, 17 bp, Artificial
  primer
cctacttcatagaaggg

SEQ ID NO: 76 - DNA, 17 bp, Artificial
  primer
aataaagccctgcagta

SEQ ID NO: 77 - DNA, 16 bp, Artificial
  primer
tgtggactctcagcag

SEQ ID NO: 78 - DNA, 17 bp, Artificial
  primer
tgtttgtgaaaagcaag

SEQ ID NO: 79 - DNA, 17 bp, Artificial
  primer
catcctgtaaaggagcc

SEQ ID NO: 80 - DNA, 15 bp, Artificial
  primer
cctgggggttttcaa

SEQ ID NO: 81 - DNA, 18 bp, Artificial
  primer
ccaccatttgctaaatct

SEQ ID NO: 82 - DNA, 16 bp, Artificial
  primer
caggtagggctcagaa

SEQ ID NO: 83 - DNA, 20 bp, Artificial
  primer
gtgctaaaagacttaaaggt

SEQ ID NO: 84 - DNA, 16 bp, Artificial
  primer
cccttgcctttttacc

SEQ ID NO: 85 - DNA, 19 bp, Artificial
  primer
cttgacctagagagtgctt

SEQ ID NO: 86 - DNA, 15 bp, Artificial
  primer
gggtccccatgaatc

SEQ ID NO: 87 - DNA, 15 bp, Artificial
  primer
tcccttgttcaggcg

SEQ ID NO: 88 - DNA, 20 bp, Artificial
  primer
aagatttttcactctgttgt

SEQ ID NO: 89 - DNA, 19 bp, Artificial
  primer
gtagaagataggaggttgg

SEQ ID NO: 90 - DNA, 20 bp, Artificial
  primer
ctaactcctgtcttaggtac

SEQ ID NO: 91 - DNA, 15 bp, Artificial
  primer
cagccaacggagcag

SEQ ID NO: 92 - DNA, 16 bp, Artificial
  primer
agcacggtctggggct

SEQ ID NO: 93 - DNA, 18 bp, Artificial
  primer
tgcctgaagtagggttgc

SEQ ID NO: 94 - DNA, 16 bp, Artificial
  primer
tgcctgccaagggtgt

SEQ ID NO: 95 - DNA, 18 bp, Artificial
  primer
ttaaggttcgggccatga

SEQ ID NO: 96 - DNA, 18 bp, Artificial
  primer
aggcgctgtttgaaggtg

SEQ ID NO: 97 - DNA, 18 bp, Artificial
  primer
gggcccaacaacactgga

SEQ ID NO: 98 - DNA, 18 bp, Artificial
  primer
aagcatctgggaccacca

SEQ ID NO: 99 - DNA, 17 bp, Artificial
  primer
ggctgtcctcaccgtca

SEQ ID NO: 100 - DNA, 18 bp, Artificial
  primer
gggctggtgagcttggtg

SEQ ID NO: 101 - DNA, 16 bp, Artificial
  primer
tgggcgatcaccgaac

SEQ ID NO: 102 - DNA, 19 bp, Artificial
  primer
tccttgctgcctgctctct

SEQ ID NO: 103 - DNA, 16 bp, Artificial
  primer
ggacaggccaatggca

SEQ ID NO: 104 - DNA, 16 bp, Artificial
  primer
ccaccgccccagactt

SEQ ID NO: 105 - DNA, 18 bp, Artificial
  primer
aggcattcgggctagctg

SEQ ID NO: 106 - DNA, 18 bp, Artificial
  primer
tttgccaaggctctctcg

SEQ ID NO: 107 - DNA, 18 bp, Artificial
  primer
ggtccggacgaactgctg

SEQ ID NO: 108 - DNA, 18 bp, Artificial
  primer
ggggatgcagagagtgga

SEQ ID NO: 109 - DNA, 18 bp, Artificial
  primer
gccagcctatgggtgtgt

SEQ ID NO: 110 - DNA, 18 bp, Artificial
  primer
aggccaggcagcagatga

SEQ ID NO: 111 - DNA, 23 bp, Artificial
  primer
agtcagaggcaggataaggatgg

SEQ ID NO: 112 - DNA, 23 bp, Artificial
  primer
cttcctgtgttccagtctctcca

SEQ ID NO: 113 - DNA, 24 bp, Artificial
  primer
tccccttactagccaggttcttgc

SEQ ID NO: 114 - DNA, 21 bp, Artificial
  primer
tggttaaccggtgtgttcctg

SEQ ID NO: 115 - DNA, 21 bp, Artificial
  primer
gagtggcctttggcctttatg

SEQ ID NO: 116 - DNA, 21 bp, Artificial
  primer
caggggcctggtcttacattc